(19) United States
(12) Patent Application Publication
(10) Pub. No.: US 2015/0004670 A1
(43) Pub. Date: Jan. 1, 2015

(54) METHODS FOR CONTINUOUS ENZYME PRODUCTION USING A FILAMENTOUS FUNGUS WITH INHIBITED GROWTH

(71) Applicant: THE BOARD OF REGENTS FOR OKLAHOMA STATE UNIVERSITY, STILLWATER, OK (US)

(72) Inventors: MICHAEL MUELLER, PIRMASENS (DE); MARK WILKINS, Stillwater, OK (US); ROLF A. PRADE, STILLWATER, OK (US)

(21) Appl. No.: 14/318,097

(22) Filed: Jun. 27, 2014

Related U.S. Application Data
(60) Provisional application No. 61/839,973, filed on Jun. 27, 2013.

(51) Int. Cl. C12N 9/24

(52) U.S. Cl. CPC C12N 9/2482
USPC 435/209

(57) ABSTRACT

Methods of continuous production of a product of interest using a filamentous fungus are provided. The fungus is auxotrophic for a nutrient that is required for fungal growth, but not for fungal synthesis of the product of interest. Under growth limiting culture conditions, in which the nutrient is absent from the culture medium, the amount of fungus remains constant or nearly constant and nutrients are not used up for growth but instead are available for synthetic pathways which produce the product of interest.

### CROSS REFERENCE TO RELATED APPLICATIONS

This application claims the benefit of U.S. Provisional Patent Application Ser. No. 61/839,973 filed on Jun. 27, 2013 and incorporates said provisional application by reference into this document as if fully set out at this point.

### STATEMENT REGARDING FEDERALLY SPONSORED RESEARCH OR DEVELOPMENT

This invention was made with U.S. Government support under USDA/NIFA Grant No. 2007-35504-18244 awarded by the Department of Agriculture. The Government has certain rights in this invention.

### FIELD OF THE INVENTION

The present invention relates generally to the field of enzyme production and, more particularly, to systems and methods of enzyme production using a filamentous fungus under zero or very low growth conditions.

### BACKGROUND OF THE INVENTION

Most biotechnological manufacturing processes use microorganisms to produce one or more metabolites of interest such as organic acids, alcohols, extracellular proteins, enzymes, etc. Because the goal is to produce the metabolite, the growth of the microorganism per se (e.g. the accumulation of biomass) is often not the priority. In fact, for these production processes, the cell mass that is produced can be considered to be a byproduct. The formation of “byproduct” cell mass uses up carbon and energy compounds that could potentially be used instead for product formation.

One such system in which biomass accumulation is a disadvantage is industrial scale enzyme production via fermentation using cultured filamentous fungi. Current technology uses mainly two fungal fermentation processes: solid state fermentation (SSF) or submerged fermentation (SmF). No other processes have proven to be applicable due to the difficulty of culturing filamentous fungi: these organisms grow as mycelia and have an affinity for surfaces, leading to clogging of pipelines during culture and manufacturing processes.

In order to address this problem, several studies have explored limiting the carbon and/or energy supplied during growth to a level at which the supplied energy is equal to the maintenance energy of the organism (Boender et al. 2009; Schrickx et al. 1993; Schrickx et al. 1995). Under these conditions, the organism approaches a zero growth rate because the energy consumed is near the maintenance ratio (Pirt 1965). Under conditions of limited nitrogen and carbon, submerged filamentous fungi cultures conidiate (Broderick and Greenshields 1981; Galbraith and Smith 1969; Ng et al. 1972).

Other studies have used retentostat cultures to analyze product formation under carbon and energy limited conditions. To achieve a retentostat, a chemostat reactor is equipped with a filter with a pore size smaller than the size of the cultured microorganism (e.g. 0.2 μm), which traps the cells in the reactor. After the desired cell mass is reached, the carbon and energy content of the continuously supplied medium is limited to reduce the metabolic rate. Jørgensen et al. (2010) reported a growth rate of Aspergillus niger approaching zero after 6 days when grown in a retentostat under carbon and energy limited conditions. However, the growth yield was still constant, leading to an increase in cell mass over time.

An observation from the study was how the percent of respired carbon changed over time (Jørgensen et al. 2010). With decreasing specific growth rate, the percent carbon respired increased. After two days, conidiation was observed, leading to an increase of melanin, which rapidly increased after four days. Stress on the cells, initiated by carbon and energy limitation, induces the transcription of the transcription factor brlA (Skromne et al. 1995), a positive regulator for conidiation (Adams and Timberlake 1990). However, results presented in these and other previous studies, (Jørgensen et al. 2010; Schrickx et al. 1993; Schrickx et al. 1995; Verseveld et al. 1991), were not conclusive with respect to whether or not continuous product formation with zero growth rate over a prolonged period of time using carbon and energy limitation is feasible. In fact, the results showed that, over the test period, the cell mass concentration increased until the end of the experiment.

Thus, what is needed are systems and methods for culturing filamentous fungus in order to produce products of interest, but which do not suffer from the disadvantages of the prior art. In particular, it would be advantageous to have available systems and methods for zero growth rate cultivation of filamentous fungi e.g. on surfaces, in order to produce products (e.g. metabolites) of interest such as proteins, enzymes, etc.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

Other embodiments and variations are certainly possible within the scope of the instant invention and can readily be formulated by those of ordinary skill in the art based on the disclosure herein.

The growth of the organisms in fermentation processes is usually not of primary interest. Growth utilizes nutrients and energy that could possibly be used for product formation. A solution for limiting or inhibiting growth of microorganisms is the reduction of carbon and energy supply to a level equal to the maintenance energy of the organism. However, control of the exact supply of carbon and energy is difficult, and often the carbon source is used for both growth and product formation.

The present invention provides novel technology to achieve low or zero growth in a culture of filamentous fungus while retaining the ability of the fungi to synthesize one or more products of interest. In an exemplary aspect, this is achieved by limiting, in the culture medium, one or more compounds for which the fungus is auxotrophic, i.e. the fungus cannot produce the substance, but the substance is necessary for fungal growth, but the one or more compounds are not required for product formation. For example, the limiting compounds are not substrates for synthesis of the product of interest. The compounds to be targeted all contain carbon, but they are not compounds used directly as energy sources. Reduction of the growth rate maximizes product formation, since substrates required for synthesis of the product of interest are still supplied but are then used for product formation and not for microbial growth. Exemplary substances which cannot be used as a carbon source for growth by filamentous fungi include, for example, co-enzymes such as pyridoxine.

The vitamin pyridoxine is necessary for fungal growth. One role of pyridoxine is as a precursor of pyridoxal-5′-phosphate (PLP), which is involved in many reactions in amino acid metabolism and synthesis. However, PLP is a trace element and is needed in only very low concentrations.

As described herein, limiting pyridoxine using Aspergillus nidulans cells that do not contain the genes necessary for pyridoxine synthesis advantageously leads to a shift of substrates away from cell growth and toward protein synthesis. Without being bound by theory, it appears that the cell matrix has enough PLP stored to perform basic maintenance reactions and to carry out protein synthesis without cell growth.

Studies described herein investigated the effects of pyridoxine limitation on the production of the enzyme xylanase B (XynB) by a culture of A. nidulans that is unable to synthesize its own pyridoxine. It was observed that when pyridoxine was absent from the medium the fungal growth was limited, but enzyme production was unaffected. Enzyme production was similar to that in a culture that grew in medium with pyridoxine, achieving 1026 U after 480 h of continuous fermentation. The growth rate of A. nidulans and the productivity of XynB production without pyridoxine limitation was also determined. A maximum growth rate of 0.311 h⁻¹ and a XynB productivity of 8.09 U/g·h was observed when pyridoxine was provided, but when the fungus was cultivated without pyridoxine, there was not cell growth and a XynB productivity of 21.1 U/g·h was observed. Thus, the level of productivity for the product of interest, in this case an enzyme, was more than twice as highunder conditions of limiting pyridoxine.

According to another aspect of the instant invention, there are provided methods and systems for continuous production of a product of interest by culturing filamentous fungi under conditions in which a substance that is necessary for fungal growth, but which is not necessary for the synthesis of a product of interest (or at least which is required in only very low quantities and/or which is not “used up” in product synthesis) is limited. Exemplary aspects include xylanase production (e.g. XynB as the product of interest or “client” enzyme) with A. nidulans under pyridoxine limitation.

The new synthesis methods have opened the door to the design of new reactor systems, since fungal growth is held in check, and clogging is thus less of an issue. Exemplary reactor systems comprise a trickle bed reactor (TBR) with recycling capabilities. Such systems utilize the advantages of both solid state fermentation (SSF) and submerged fermentation (SmF). In this aspect of the invention, the fungus is allowed to grow on a solid inert support, and medium containing carbon and an energy source trickles down onto the fungi. Uncontrolled growth of mycelia in the tubing of the apparatus, which would eventually otherwise lead to clogging, is substantially reduced by utilizing a growth media that does not include pyridoxine, i.e. the fungus is grown under conditions of pyridoxine limitation. This fungus is unable to synthesize its own pyridoxine and is unable to grow when no pyridoxine is present in the medium, but enzyme production is unaffected. The current study demonstrated successful continuous operation over 18 days with high XynB titers. The reactor achieved a XynB output of 41 U/ml with an influent and effluent flow rate of 0.5 ml/min and a recycle flow rate of 56 ml/min. Production yields were 1.4 times higher than a static tray culture and between 1.1 and 67 times higher compared to SSF enzyme production described in the literature.

The foregoing has outlined in broad terms the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

Other objects and advantages of the invention will become apparent upon reading the following detailed description and upon reference to the drawings in which:

FIG. 1 is a table showing total cell mass, protein concentration, substrate concentration (glucose+glucose equivalent from maltose), specific growth rate and yields for A. nidulans at different initial substrate concentrations, with standard deviation of two replicates.

FIG. 2 shows XynB production over time with limited pyridoxine and 48 h transplanting frequency. The culture was initially (0-48 h) grown on medium with pyridoxine and pyridoxine was added again at 288 h. Error bars represent standard deviation of two replicates.

FIG. 3 shows XynB production over time with limited pyridoxine and 24 h transplanting frequency. The culture was initially (0-72 h) grown on medium with pyridoxine and pyridoxine was added again at 144 h. Error bars represent standard deviation of two replicates.

FIGS. 4A and B shows maltose (A) and glucose (B) utilization during enzyme production with limited pyridoxine with 48 h transplanting frequency.

FIGS. 5A and B shows maltose (A) and glucose (B) utilization during enzyme production with limited pyridoxine with 24 h transplanting frequency.

FIGS. 6A and B shows pH development over time for 48 h (A) and 24 h (B) transplanting frequency. Error bars represent standard deviation of two replicates.

FIG. 7 depicts a schematic of an exemplary trickle bed reactor design.

FIG. 8 shows the effect of fermentation mode on XynB production over time at 37° C. Tray and shaken flask fermentations had the same volume of medium and inoculum concentration. Error bars represent standard deviation of two replicates.

FIG. 9 shows an SDS-PAGE analysis of proteins produced by A. nidulans in flask and tray cultures. 1=Flask 48 h, 2=Flask 72 h, 3=Flask 96 h, L=Ladder, 4=Tray 48 h, 5=Tray 72 h, 6=Tray 96 h. “Ladder” is the lane with standards.

FIGS. 10 A and B shows (A) A. nidulans pellets grown in a shaking flask culture at 37° C. for 96 h rotated at 225 rpm and (B) shaking flask fermentation broth grown after 96 h.

FIG. 11 is a comparison of residence time with different recycle flow rates through lava rock packing of a trickle bed reactor. Testing was performed with DI water at room temperature.

FIGS. 12A and B depicts graphs showing protein formation (A) and substrate utilization (B) in a trickle bed reactor and storage container without pyridoxine limitation using semi-continuous operation. Every 6 h, 240 ml of fermentation broth were pumped out and replaced with fresh medium.

FIGS. 13A and B depicts graphs showing XynB Production and pH development (A) and substrate utilization (B) with 1.0 ml/min influent and effluent flow rate. Recycle flow rate was 224 ml/min.

FIG. 14 depicts a graph showing XynB production and pH development in trickle bed reactor. Continuous flow in and out was set at 0.5 ml/min. Initial recycle flow rate was 224 ml/min.

FIG. 15 shows an SDS-PAGE analysis of reactor XynB production at different selected time points (indicated in days). “Ladder” is the lane with standards.

FIG. 16 is a graph comparing maltose and glucose consumption in a trickle bed reactor. Continuous flow in and out was set at 0.5 ml/min. The initial recycle flow rate was 224 ml/min.

FIGS. 17A and B are graphs showing a comparison of actual XynB production in a reactor (A) and substrate utilization (B) to predictions using CSTR modeling. With S₀=57.6 g/L, Y_{P/X}=480 U/g, Y_{X/S}=1.13, μₙₑₜ=0.061 h⁻¹ (Model 1), and μₙₑₜ=0.164 h⁻¹ (Model 2). μₙₑₜ for Model 2 was back calculated from actual product formation in reactor. The cell concentration is assumed constant after initiating continuous fermentation.

FIG. 18 is a graph showing a comparison of actual cumulative XynB content in a reactor and a model prediction using an experimentally determined productivity of 21.14 U/g dry cell mass*h and a cell mass prediction using CSTR modeling.

FIG. 19A-C shows the fungal growth in a trickle bed reactor on a lava rock surface. Growth after 1 day (A), 2 days (B), and 9 days (C).

FIG. 20 shows a double reciprocal plot of μnet and initial substrate concentration S with A. nidulans grown in Petri dishes.

FIG. 21 shows a plot of a model prediction of cell mass in a reactor, Model 1 with μₙₑₜ=0.061 h⁻¹ and Model 2 with μₙₑₜ=0.164 h⁻¹.

FIGS. 22A and B shows a graphical comparison of actual maltose (A) and glucose (B) utilization to predictions using CSTR modeling for an embodiment of the invention. With S₀_{_{—}}ₘₐₗₜₒₛₑ=47.6 g/L, S₀_{_{—}}_{Glucose}=10 g/L, Y_{P/X}=480 U/g, Y_{X/S}_{_{—}}_{Maltose}=1.2, Y_{X/S}_{_{—}}_{Glucose}=19.2, μₙₑₜ=0.061 h⁻¹ (Model 1), and μₙₑₜ=0.164 h¹ (Model 2). μₙₑₜ for Model 2 was back-calculated from actual product formation in a reactor. The cell concentration is assumed constant after initiating continuous fermentation.

### DESCRIPTION OF EMBODIMENTS

Methods and systems for continuous production of a product of interest by culturing filamentous fungi are provided. The methods involve limiting the growth rate of the fungus to zero or near zero by eliminating from the culture medium one or more substances that are required for fungal growth (cell division, reproduction, increase in cell mass, etc.) but which are not “used up” (e.g. are not incorporated into) a product of interest that is synthesized by the fungus. The one or more substances may be used in synthesis of the product of interest, but in this case, they function as e.g. catalysts, co-factors, etc. which may be reused or recycled by the cell, rather than being incorporated into or becoming part of the product of interest, i.e. the substance is not “used up” by the formation of the product of interest. Thus, synthesis of the product of interest does not deplete the supply of the limiting substance, or synthesis of the product of interest does not require the presence of the limiting substance. In other words, the amount of the substance(s) in the culture medium is/are maintained at a level that is sufficient to maintain the fungal cells (e.g. to maintain their capacity to synthesize the product of interest), but that is not sufficient to promote fungal cell growth. The level of substance(s) in the culture medium may be extremely low or may be zero. For example, the presence of the substance(s) may be due only to storage of the substance(s) by the fungus during a previous growth phase. Typically, the substance(s) is/are not added, or are added only intermittently and at extremely low levels, to the culture medium that is used during the fungal maintenance/product synthesis stage of the production process.

As a result of these improved product synthesis methods, under limiting conditions as described herein, the yields of the one or more products of interest are increased, compared to yields obtained when the one or more limiting substances are provided. For example, the yield may be increased e.g. by at least about 2, 5, 10, 15, 20, 25, 30, 35, 40, 45, 50, 55, 60, 65, 70, 75. 80. 85, 90, 95 or 100 fold or more. Accordingly the invention also provides methods of increasing the yield of a product of interest that is synthesized by a filamentous fungus, e.g. an auxotrophic filamentous fungus, by limiting, in the growth media of the fungus, the substance or substances for which the fungus is auxotrophic. In other words, production yield of one or more products of interest is increased by reducing the growth rate of the fungus to zero or near zero, while maintaining the synthetic capability of the fungus (at least with respect to the one or more products of interest).

By “zero or near zero” we mean that either no measurable growth/production of fungal biomass is detectable, or that at most the specific rate of growth and/or increase in biomass is less than about 0.01 h⁻¹, e.g. is in the range of from about 0 to about 0.01 h⁻¹.

By “zero or extremely low” concentration(s) of substance(s) we mean that a substance that is limited is present in the culture medium at a concentration of less than about 0.0009 g/L, e.g. in the range of from about 0 to about 0.0009 g/L for pyridoxine. These concentrations may differ for other substances and will be determined by techniques described herein.

By limiting the “growth” of the fungus, we mean that one or both of cell division (reproduction) and/or the accumulation of cell mass (of individual cells) is halted or at least reduced to an extremely low level, e.g. to less than a rate of 0.01 h⁻¹. The invention employs filamentous fungi to produce one or more products of interest. Such fungi may be native fungi, or they may be mutants that are naturally occurring and selected, or genetically engineered to be unable to produce, or to produce in very low amounts, a substance that is necessary for growth. In other words, the fungi are auxotrophic for the substance. However, growth under conditions of substance limitation as described herein does not have a deleterious effect on the ability of the fungus to synthesize at least one product of interest. Exemplary filamentous fungi that are used in the practice of the present invention, any of which are selected to be auxotrophic or are rendered auxotrophic via mutation include but are not limited to: Aspergillus species such as A. nidulans, A. japonicus, A. nidulans, A. niger, A. oryzae, A. fumigatus, A. fumigatus var. niveus, A. flavus, A. sojae, A. terreus; Ceriporiopsis species such as C. subvermispora Chrysosporium species such as C. lucknowense; Myceliophthora species such as M. thermophile (Sporotrichum thermophilum), Thielavia heterothallica; Penicillium species such as P. canescens, P. citrinum, P. decumbens, P. funiculosum, P. janthinellum, P. oxalicum, P. janczewskii and P. solitum; Phanerochaete species such as P. chrysosporium; Rhizopus species such as Rhizopus oryzae, Rhizopus stolonifer, Rhizopus arrhizus, and Rhizopus oligosporus; Talaromyces species such as T. flavus; and Trichoderma (Hypocrea) species such as T. reesei, T. viride, T. harzianum, and T. virens.

In some aspects, the fungal strain itself is also mutated or selected for its ability to produce a desired amount of a product of interest while its growth is limited by coenzyme limitation. For example, fungi that cannot produce at least one nutrient which is required for growth, but is not required for formation of a product of interest, may be made via genetic engineering or by selection using known techniques. Alternatively, or in addition, the fungus may be engineered or selected to overproduce the one or more products of interest.

Substances that may be limited during a maintenance/synthesis culture phase (during which the fungi do not grow but do synthesize at least one product of interest), include but are not limited to: various vitamins and/or the cofactors and/or prosthetic groups which they form, for example: Thiamine (B1); Thiamine pyrophosphate; Niacin (B3); NAD+ and NADP+; Pyridoxine (B6); Pyridoxal phosphate; Lipoic acid; Lipoamide; Methylcobalamin; Cobalamine (B12); Cobalamine; Biotin (H); Pantothenic acid (B5); Coenzyme A; Folic acid (B9); Tetrahydrofolic acid; Vitamin K; Menaquinone; Vitamin C; Ascorbic acid; Riboflavin (B2); Flavin mononucleotide; Flavin adenine dinucleotide; Coenzyme F420; etc.

Non-vitamin co-factors that may be limited include but are not limited to: Adenosine triphosphate, S-Adenosyl methionine, Coenzyme Q, Cytidine triphosphate, Glutathione, Heme, Molybdopterin, Nucleotide sugars, 3′-Phosphoadenosine-5′-phosphosulfate, Pyrroloquinoline quinine, Tetrahydrobiopterin, Tetrahydromethanopterin, etc.

Other substances that may be limited include but are not limited to: one or more amino acid residues which are necessary for fungal growth but which are not necessary for synthesis of the product of interest; or one or more inorganic co-factors such as metal ions which are necessary for fungal growth, but which are not necessary for synthesis of the product of interest (e.g. manganese, magnesium, copper, iron, molybdenum, nickel, zinc, or iron-sulfur clusters).

The general culture conditions of the filamentous fungi are those known to those of skill in the art. Typically, a seed culture is obtained and expanded in a desired volume to a desired concentration of fungal cells using a culture medium that is not limiting. The time of this culturing phase is generally in the range of from about 24 to about 168 hrs, e.g. about 1, 2, 3, or 4 days, or possibly longer. Once a desired concentration of cells is achieved, the medium is changed or switched and/or the fungi are removed from the growth medium and placed in a different, limiting medium. In the limiting medium, one or more substances that are required for further growth and/or mass accumulation of individual fungal cells is limited, so that the numbers of cells and/or the size and weight of individual fungal cells remains constant or increases insignificantly, e.g. at a rate that does not impact production of the product of interest. Typically, the “growth” rate is zero or near zero. However, the cells continue to carry out metabolic activities, including synthesis of the one or more products of interest. The fungi are generally cultured under these conditions from a period of time, e.g. from about 24 h to about 72 h, and usually at least about 48 h.

Exemplary media that can be used to culture the fungi include but are not limited to carbon sources such as glucose, maltose, xylose, mannose, fructose, arabinose, sucrose, wheat bran, and oat bran; nitrogen sources such as ammonia, ammonium salts, corn steep liquor, yeast extract, cotton seed extract, peptone, yeast nitrogen base and malt extract; phosphate salts, vitamins such as ribloflavin, pyridoxine, biotin, vitamin B₁₂ and other B vitamins; and various metal salts and salt solutions such as Clutterbuck salt solution and trace metal solution, etc. Growth conditions are typically in the range of e.g. a pH in the range of from about 4 to about 8; a temperature in the range of from about 10 to about 40° C.; an oxygen concentration of from about 0% to about 21%; and water activity from 0.7 to 1.0.

Products of interest that may be synthesized by the fungi during this maintenance/synthesis stage or phase include but are not limited to: various proteins, e.g. enzymes such as xylanases, pectinases, glucosidases, amylases, phytases, proteases, transacetylases, ligases, esterases, hydrolases, and other enzymes produced by fungi; non-enzyme proteins such as antibiotics, therapeutic proteins and animal feed supplements; other products of interest such as amino acids, alcohols, organic acids, lipids, hydrocarbons, aldehydes, esters and other organic molecules.

Those of skill in the art are familiar with many types of reactors that may be used for the culture of filamentous fungi as described herein. Exemplary culturing systems include but are not limited to: trickle bed reactors, packed bed reactors, submerged fermenters, and static tray reactors.

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

### EXAMPLES

### Example 1

### Culture of Aspergillus nidulans Under Conditions of Limiting Pyridoxine

The growth of the organisms in fermentation processes is usually not of primary interest. Growth utilizes nutrients and energy that could possibly be used for product formation. The current solution for limiting or inhibiting growth of microorganisms is the reduction of carbon and energy supply to a level equal to the maintenance energy of the organism. However, control of the exact supply of carbon and energy is difficult, and often the carbon source is used for both growth and product formation. A different approach to limiting growth can be the limitation of co-enzymes. The present study investigated the limitation of pyridoxine on an Aspergillus nidulans culture unable to synthesize its own pyridoxine that produces xylanase B (XynB) as client enzyme. It was observed that the growth was limited when pyridoxine was absent, while the enzyme production was unaffected. The enzyme production was similar to a culture that grew on medium with pyridoxine and achieved 1026 U after 480 h of continuous fermentation. Furthermore, the present study investigated the growth rate of A. nidulans and determined the productivity of XynB production under pyridoxine limitation. No fungal growth and a XynB productivity of 21.14 U/g·h were observed under pyridoxine limitation. A maximum growth rate of 0.311 h⁻¹ and a XynB productivity of 8.09 U/g·h was observed when pyridoxine was provided.

Industrial scale enzyme production with filamentous fungi uses mainly two fermentation processes: solid state (SSF) or submerged fermentation (SmF). No other process has proven to be applicable due to the difficulty of culturing filamentous fungi. Filamentous fungi grow as a mycelium, which can lead to clogging of pipelines due to their affinity to surfaces.

Most biotechnological processes are used for the production of metabolites (e.g. organic acids, alcohols, extracellular proteins, etc.). The growth of the organism and, therefore, the accumulation of biomass, is often not the priority. For these processes, the cell mass that is produced can be considered a byproduct. The formation of cell mass uses carbon and energy compounds that could potentially be used for product formation. In many studies it was proposed to limit carbon and energy supply in chemostats to a level where the supplied energy is equal to the maintenance energy of the organism (Boender et al. 2009; Schrickx et al. 1993; Schrickx et al. 1995). Then, the organism will approach a zero growth rate when the energy consumed is near the maintenance ratio (Pirt 1965). It was shown that submerged filamentous fungi cultures conidiate when nitrogen and carbon are limited (Broderick and Greenshields 1981; Galbraith and Smith 1969; Ng et al. 1972). Previous studies used retentostat cultures to analyze product formation under carbon and energy limited conditions. To achieve a retentostat, a chemostat is equipped with a filter with a pore size smaller than the size of the organism (0.2 μm), which traps the cells in the reactor. After the desired cell mass is reached, the continuously supplied medium has limited carbon and energy content to reduce the metabolic rate. Jørgensen et al. (2010) reported a growth rate of A. niger approaching zero after 6 days when grown in a retentostat under carbon and energy limited conditions. However, the growth yield was constant, leading to an increase in cell mass over time. An interesting observation from the study was how the percent of respired carbon changed over time (Jørgensen et al. 2010). With decreasing specific growth rate, the percent carbon respired increased. After two days conidiation was observed, leading to an increase of melanin, which rapidly increased after four days. Stress on the cells, initiated by carbon and energy limitation, induces the transcription of brlA (Skromne et al. 1995). The transcription factor brlA is a positive regulator for conidiation (Adams and Timberlake 1990). From the results presented in previous studies, (Jørgensen et al. 2010; Schrickx et al. 1993; Schrickx et al. 1995; Verseveld et al. 1991), it is not conclusive if continuous product formation with zero growth rate over a prolonged period of time using carbon and energy limitation is feasible. The results show that over the test period the cell mass concentration increases until the end of the experiment. Additionally, no literature on zero growth rate experiments was found with filamentous fungi grown on surfaces. Furthermore, no literature was found initiating a zero growth rate by limiting other essential nutrients in the medium that do not supply the organism with carbon or energy. Examples could be the limitation of vitamins or coenzymes necessary for certain cell functions.

Reducing the growth rate can maximize product formation, since the substrate is used for product formation and not for microbial growth. A potential compound present in the medium necessary for fungal growth is pyridoxine. Pyridoxine is a vitamin and precursor for pyridoxal-5′-phosphate (PLP), which is involved in many reactions in amino acid metabolism. One reaction is transamination, where an α-amino acid is converted to an α-keto acid (Voet and Voet 2004). PLP acts as a coenzyme together with the enzyme in the form of a Schiff base. It is also involved throughout amino acid biosynthesis in the conversion of aspartate to lysine, threonine to methionine and α-ketoisocaproate to leucine. PLP is a trace element and only needed in very low concentrations. It is proposed that a limitation of pyridoxine on an A. nidulans culture with a pyridoxine marker in the fermentation medium would lead to a shift of substrates to protein formation. The cell matrix has enough PLP stored to perform basic reactions and protein formation without growth. No literature has been found where the limitation of pyridoxine was tested on filamentous fungi.

Materials and Methods

### Strain and Spore Formation

Aspergillus nidulans with the number 733 in the Fungal Genetic Stock Culture (FGSC) was used to conduct the experiment. The strain has a pyridoxine marker. The modification of A. nidulans to express Xylanase B as a client protein is described elsewhere (Segato et al. 2011). Spores were kept in a 20% glycerol, 10% lactose solution as a stock culture at −80° C. Spores were made by using a solid medium containing 9.0 g/L glucose, 50 ml/L 20× Clutterbuck salt solution (120 g/L NaNO₃, 10.4 g/L KCl, 10.4 g/L MgSO₄, 30.4 g/L KH₂PO₄), 1 ml/L 1000× trace element solution (22 g/L ZnSO₄.7H₂O, 11 g/L H₃BO₃, 5.0 g/L MnCl₂.7H₂O, 5.0 g/L FeSO₄.7H₂O, 1.6 g/L CoCl₂.5H₂O, 1.6 g/L CuSO₄.5H₂O, 1.1 g/L Na₂MoO₄.4H₂O, 50 g/L Na_{z}-EDTA), 1 ml of a 1 g/L pyridoxine solution, and 15% agar. The pH was adjusted to 6.5 using 10 N NaOH. Twenty μl of stock spore solution were distributed on the agar surface. The plates were kept for 48 h at 37° C. After incubation the plates were stored at 4° C. until usage.

### Fermentation Medium

The fermentation medium contained 47.6 g/L maltose and 9.0 g/L glucose as carbon sources. Maltose is necessary to activate the promoter for Xylanase B expression. The medium also contained 50 ml/L 20× Clutterbuck salt solution, 1 ml/L 1000× trace element solution, and 1 ml of a 1 g/L pyridoxine solution. For the maximum growth rate experiment, maltose and glucose concentrations were varied, but were kept at the same ratio (4.7:1) according to best protein formation with 47 g/L maltose and 10 g/L glucose. The pH was adjusted to 6.5 using 10 M NaOH. The medium was sterilized by autoclaving at 121° C. for 20 min.

Determination of Maximum Growth Rate of A. nidulans

Before the maximum growth rate (μₘₐₓ) can be obtained, multiple experiments are necessary to investigate the specific growth rate (μₙₑₜ) at different initial substrate (S) concentrations. The Monod model was used as the underlying base for μₘₐₓ and μₙₑₜ determination. For μₙₑₜ it is required to measure cell mass concentration over time. Since the organism is a filamentous fungus, representative sampling is not possible. To overcome this problem, multiple parallel tests were started with different inoculation times. The tests were carried out in Petri dishes. Each dish was filled with 20 ml of medium with identical substrate concentration. All parallel tests were inoculated with the same amount of spore solution (20 μl) with the same spore concentration. Two dishes were inoculated for each time interval of 12, 24, 36 and 48 h. After these time intervals, the complete cell mycelium was harvested, washed with deionized water and dried at 50° C. for one day. The protein concentration in the broth was analyzed by the Bradford assay (Bradford 1976). An aliquot of broth was used for sugar analysis with HPLC. The logarithm of dry cell mass was plotted vs. time. The slope of the linear regression in the linear region of the plot represented μₙₑₜ. This process was repeated for different initial maltose and glucose concentrations. The obtained μₙₑₜ was plotted in a reciprocal form vs. 1/S. For this plot S represents the glucose concentration plus the glucose equivalent of maltose. A linear regression led to μₘₐₓ and K_{S}, which were 0.311 h⁻¹ and 1.34 g/L, respectively.

Evaluation of XynB Production with A. nidulans Under Pyridoxine Limitation

Two tests with different transplanting frequencies, 24 h and 48 h, were chosen. To grow a mycelium, a Petri dish was filled with 20 ml of medium and inoculated with 20 μl of spore solution. The culture was allowed to grow at 37° C. until a mycelium covered the surface of the medium. After inoculation part of the mycelium was manually transferred into a Petri dish previously filled with medium lacking pyridoxine. This culture was allowed to grow either 24 h or 48 h and then again transferred into medium without pyridoxine. This was repeated until protein production decreased. Then, the mycelium previously grown on pyridoxine-free medium was transferred into medium with pyridoxine and after either 24 h or 48 h it was again transferred into medium without pyridoxine. The cultures were transferred nine and five times for the 48 h and 24 h culture, respectively. After each transfer interval, the protein concentration was measured by the Bradford assay, the activity was determined with DNS reagent, the wet mass of transferred mycelium was obtained, the pH was measured and the sugar and organic acid concentration were measured with HPLC. Additionally to both tests, a control mycelium, which was always placed into medium with pyridoxine, was evaluated. Both test and control were evaluated in duplicate.

Determination of A. nidulans XynB Productivity Under Pyridoxine Limitation

In order to obtain the enzyme productivity under pyridoxine limitation, a mycelium had to be grown on medium with pyridoxine and then transferred into medium without pyridoxine. A Petri dish was filled with 20 ml medium with pyridoxine and was inoculated with 20 μl of spore solution. The culture was grown for 48 h at 37° C. After 48 h the culture was transferred into medium without pyridoxine and after 48 more h it was again transferred into medium without pyridoxine. After this second transfer, the mycelium growth was examined based on visual comparison before and after incubation. After 24 h the medium was analyzed for sugar concentration with HPLC, protein concentration with Bradford assay and xylanase activity with DNS reagent. The mycelium was washed with DI water and dried for one day at 50° C. to obtain the dry cell mass. The productivity was determined by calculating the total activity (U) from the volumetric activity (U/ml) and dividing it by the total dry cell mass (g) and 24 h. The enzyme activity was measured to determine the existence of XynB. The test was performed in triplicate and compared to a control culture that grew on medium with pyridoxine.

### Determination of Protein Concentration

The protein concentration was determined using a Bradford assay. Forty μl of Bradford coommassie solution was pipetted into a well of a 96-well plate. In order to stay within the absorbance calibration curve limit of 2.5 at 575 nm, the amount of enzyme solution varied depending on the protein concentration received from the test. DI water was added to achieve a total volume of 200 μl. The blank well contained only 40 μl Bradford solution and 160 μl DI water. The absorption was measured using a UV-Vis 96-well plate reader (Tecan Infinite M200, Männedorf, Switzerland) at 595 nm.

### Determination of XynB Activity

XynB was analyzed for its specific activity on xylan from beechwood (Sigma Aldrich). Forty-nine μl of 0.5% substrate solution prepared with 25 mM ammonium acetate buffer at pH 6 and 1 μl of enzyme solution were added into a well of a 96-well plate. The plate was incubated in a water bath at 50° C. for 15 min. After incubation, the 96-well plate was removed from the water bath and 50 μl of dinitrosalicylic acid (DNS) reagent was added immediately to terminate the enzymatic hydrolysis. The 96-well plate was placed in a second water bath at a temperature of 100° C. for 5 min to achieve the color formation. After the reaction time, 100 μl of the liquid was transferred in a 96-well reading plate and analyzed at 575 nm for reducing sugar concentration. With the following equation the specific activity was calculated based on the spectrophotometer values:

$$U = ( ( ( A - 0.047 ) / F ) * ( \frac{{V}_{assay}}{t } ) ) / {p}_{enzyme}$$

where U=specific activity [μmol of reducing sugar/mg protein·min], A=absorbance at 575 nm, F=calibration factor, V_{assay}=assay volume (200 μl), t=incubation time [min] and p_{enzyme}=mass protein in enzyme used [mg].

In this embodiment, the final results are given in XynB activity units per volume, U_{V},

U_{V}=U·C_{P}

with C_{P}=protein concentration in mg/ml.

### Sugar and Organic Acid Analysis

Concentrations of glucose and maltose were analyzed on an HPX-87P column (Bio-Rad, Sunnyvale, Calif., USA). The eluent was HPLC grade DI-water with a flow rate of 0.6 ml/min at 85° C. and a refractive index detector (1100 Series Agilent, Santa Clara, Calif., USA) (Sluiter et al. 2008c). Organic acids were analyzed on a HPX-87H column (Bio-Rad, Sunnyvale, Calif., USA). The eluent was 0.01 N sulfuric acid with a flow rate of 0.6 ml/min at 60° C. A refractive index detector (1100 Series Agilent, Santa Clara, Calif., USA) was used for detection (Sluiter et al. 2008c).

### Results and Discussion

Growth Rate of A. nidulans

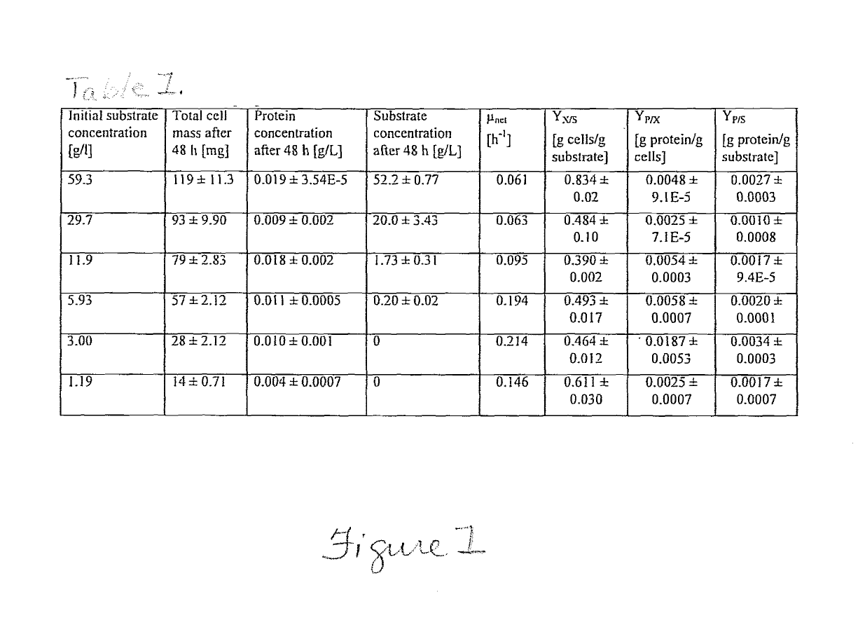

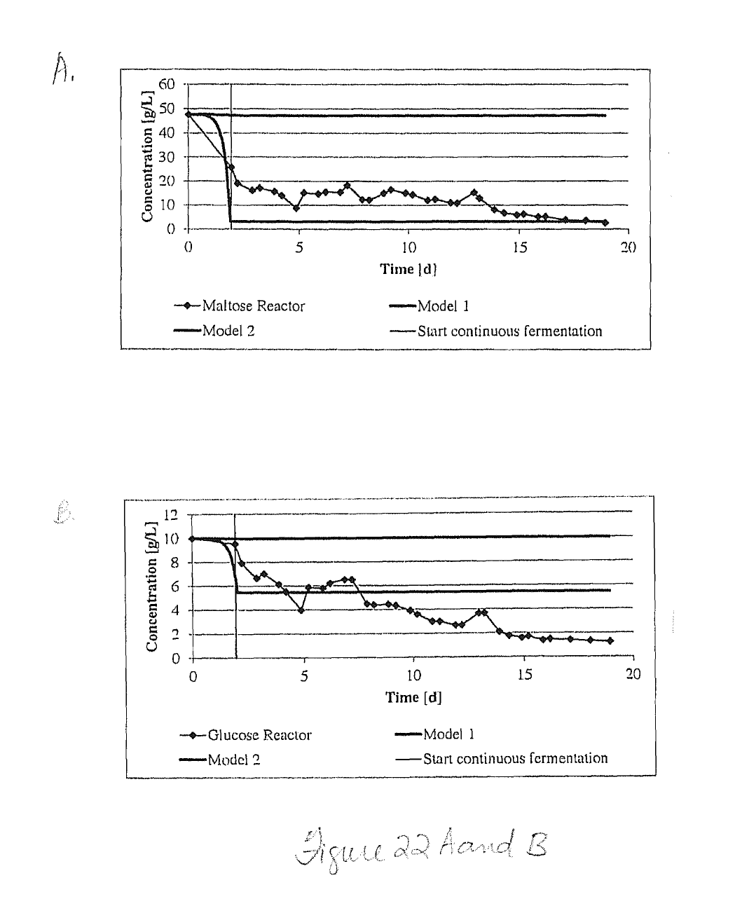

Most of the literature that deals with analyzing growth kinetics of filamentous fungi either use submerged cultures or cultures grown on solid medium or surfaces. When grown as a surface culture, the parameters for growth rate are based on circumferential growth, rather than on a mass per volume unit. The present study uses a surface grown culture, but applies the same methodology as for submerged cultures. Table 1 depicted in FIG. 1 shows the total cell mass, protein and substrate concentrations after 48 h, as well as the specific growth rates for the corresponding initial substrate concentrations and yields. Using parallel tests to imitate sampling at different time points worked well for this system and the growth curves showed the familiar pattern observed for many microorganisms. The total cell mass after 48 h increased with increasing initial substrate concentration. However, it was found that μₙₑₜ does not increase with substrate concentration. In fact, a typical substrate inhibition pattern in the double reciprocal plot of μₙₑₜ versus S was found (FIG. 20). Hence, the fungus grows at a slower rate at higher substrate concentrations.

The highest μₙₑₜ with a value of 0.214 h⁻¹ was found with initial substrate concentrations of 2.4 g/L maltose and 0.45 g/L glucose. At higher substrate concentrations, the fungus indeed grows more slowly, but continuously for at least 48 h. At lower substrate concentrations, the fungus grows rapidly to exploit all nutrients, but stops growing when the nutrients are depleted. The maximum specific growth rate is 0.311 h⁻¹, and K_{S} was 1.33 g/L.

It was found that the mycelia of filamentous fungi develop differently for different substrate concentrations in the environment. At high substrate concentration, the hyphae form a dense network with more branches per area. The purpose is to utilize more efficiently the nutrients in that area. When the substrate concentration is low, the hyphae branch less per area and grow longer in order to reach a location with higher substrate concentration (Prosser and Tough 1991). This behavior was also observed in the present study. The cultures with low initial substrate concentration were more like a gel throughout the liquid; whereas, the cultures with high initial substrate concentration developed a mycelium layer on top of the surface of the liquid at the end of the test.

For industrial applications a growth rate based on mass mycelium per time is more useful than a growth rate based on circumferential growth. In technical applications it is often not practical to measure circumferential growth. Cultures that grow in nutrient rich medium also become thicker (more mass per area), which would not be included in measuring circumferential growth.

XynB Production with Inhibited Growth

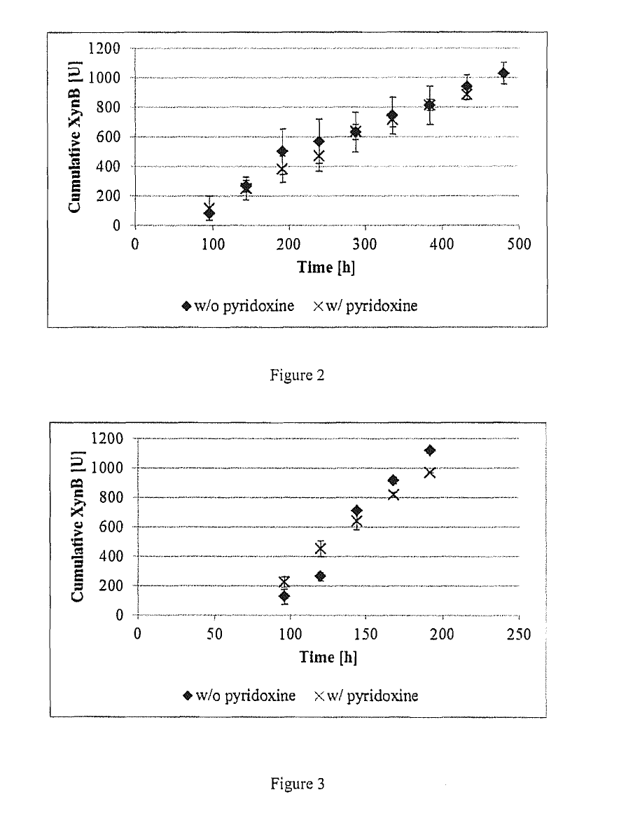

It was observed that the fungus produces the desired enzyme, XynB, even when growth is suppressed. FIG. 2 shows the total accumulative XynB activity over time for the experiment with a transplanting frequency of 48 h. The average XynB activity was 10.0±5.13 U/ml for the culture grown without pyridoxine. The control showed an average XynB activity of 9.78±3.0 U/ml. It was observed that the protein concentration drops to 0.08 g/L after 192 h when grown on medium without pyridoxine. The 240 h and 288 h samples showed a yellow colored broth that smelled similar to urine. In these samples the pH dropped from 8.5 to 4.5. After 288 h the culture was transferred into medium with pyridoxine to recover. At 336 h, no sign of smell was observed and the broth color was again the normal amber color. However, even though the protein concentration was low during 240 h and 288 h, the activity was still comparable to the previous levels. This indicates a higher proportion of xylanase B in the total protein mix. No difference in XynB production was observed between the test without pyridoxine and the control.

With a transplanting frequency of 24 h, the average XynB activity was 9.63±5.64 U/ml and 9.88±2.06 U/ml for the culture grown without and with pyridoxine, respectively. FIG. 3 shows the cumulative XynB activity over time. The phenomenon of decreased protein formation with a simultaneous increase in activity, as was found for both transplanting frequencies, shows that XynB formation is independent of mycelium growth. Furthermore, it can be seen in FIG. 3, that when the culture was transplanted with a 24 h frequency, the accumulative XynB production reaches the same levels as the culture with 48 h transplanting frequency, but at 192 h as opposed to 480 h. Hence, it is not necessary to use a 48 h frequency, since the same XynB activity is already reached after 24 h. XynB production was higher for the test without pyridoxine compared to the control when a transplanting frequency of 24 h was used.

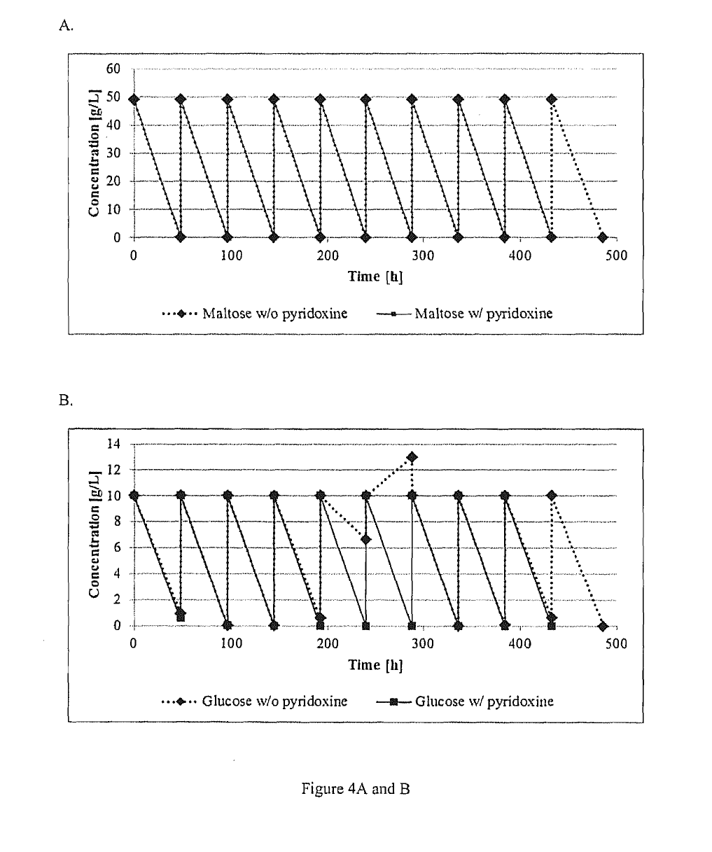

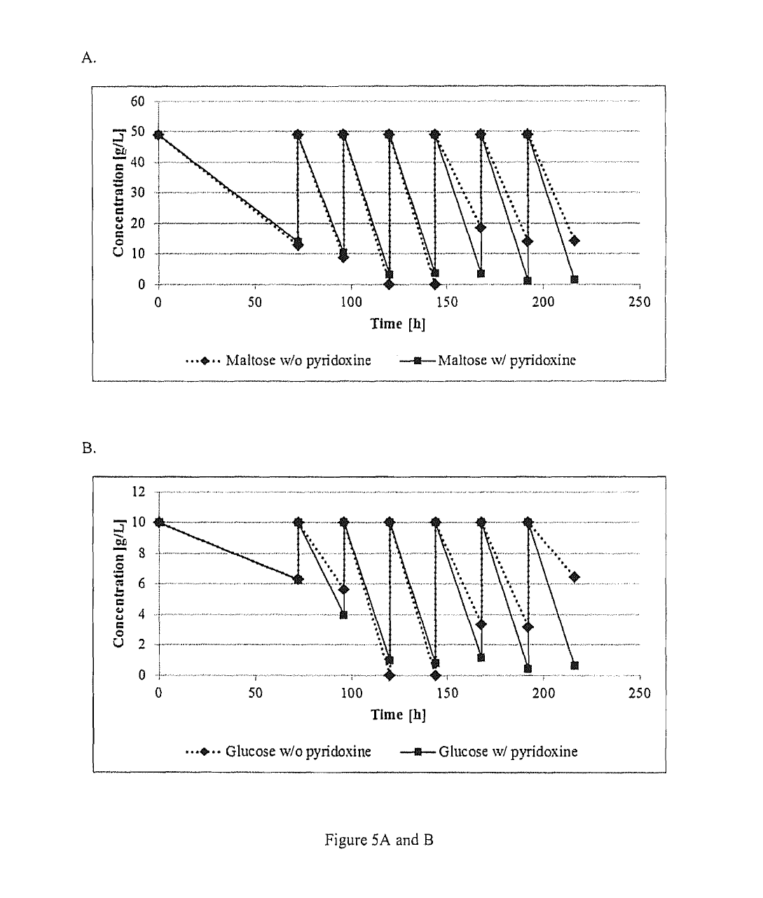

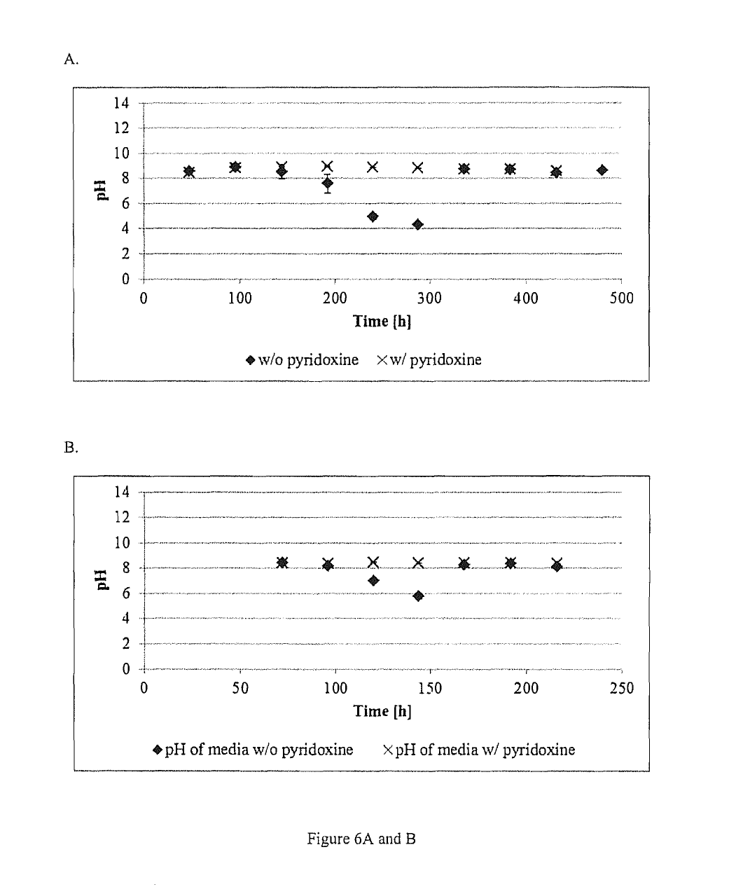

Substrate utilization is shown in FIGS. 4A and B. At all times maltose (A) was completely utilized for 48 h transplanting frequency. Glucose (B) was always utilized except at 240 h and 288 h when grown on medium without pyridoxine. At 288 h the glucose concentration increased to 12.99 g/L, indicating that the metabolism still utilizes all maltose and converts some of it to glucose, which is then not used further. With a 24 h transplanting frequency, the average residual maltose and glucose concentration was 11.39 g/L and 4.98 g/L, respectively, when grown on medium without pyridoxine (FIGS. 5A and B). The maltose and glucose utilization was higher for the culture grown on pyridoxine than for the culture grown on medium without pyridoxine. The control showed average maltose and glucose concentrations of 5.30 g/L and 2.03 g/L, respectively. Compared to the culture grown without pyridoxine, the control showed new mycelium growth, which requires substrate and explains the faster nutrient utilization. After 192 h pyridoxine needs to be replenished for further enzyme production. The moment of pyridoxine replenishment can be monitored by measuring pH. It is recommended to replenish pyridoxine before the pH drops below 6 in order to avoid a decrease in protein production (FIGS. 6A and B). The positive correlation of pH to protein production is a useful tool for reactor control and monitoring. Whereas online monitoring of pH is ready available, online measurement of protein concentration is more difficult.

No measurement of cell mass was performed due to the impossibility of taking homogenous samples. Pictures were taken to document the constant surface area of the fungus throughout the experiment. No visual growth of surface area (enlargement of mycelia) took place when the fungus was kept on medium without pyridoxine. The fungus's color became darker while using medium without pyridoxine.

The cause for the pH decrease over time under pyridoxine limitation appears to be linked to succinic acid production. An increase of succinic acid from 0.06 g/L to 0.26 g/L could be observed when the pH dropped between 240 h and 288 h and decreased back to 0.08 g/L at 336 h (data not shown). It is expected that pyridoxine limitation causes an increase in organic acids and/or amino acids due to the involvement of pyridoxine in amino acid metabolism. Other organic acids and amino acids either were not observed to increase or not detected with HPLC.

Besides of the changes that occur in the metabolism based on pyridoxine limitation, fungal autolysis can also be responsible for the pH decrease. Pyridoxine indirectly leads to starvation by disturbing the amino acid metabolism, which has been shown to initiate autolysis (White et al. 2002). Autolysis is a process in which the fungus enzymatically breaks down old fungal mass, in order to utilize the nutrients for fungal hyphae tip growth. This breakdown of fungal mass could release compounds responsible for pH decrease.

Productivity of A. nidulans with Inhibited Growth

Table 2 shows all factors obtained from this experiment. The goal was to determine the protein productivity of an A. nidulans culture when initially grown on medium with pyridoxine and then transferred onto medium without pyridoxine. The results presented are obtained after transplanting the culture a second time. After the second transplantation, no visual growth was observed with the culture without pyridoxine. The productivity was obtained and had a value of 21.14 U/g dry cell mass*h for the culture without pyridoxine and 8.09 U/g dry cell mass*h for the culture with pyridoxine. The productivity without pyridoxine was 2.6 times higher compared to the productivity with pyridoxine over the same time period, which is also represented in the yields. The protein to dry cell mass yields (Y_{P/X}) of the culture without pyridoxine and the culture with pyridoxine were 507.42 U/g and 194.21 U/g, respectively. The protein to substrate yield (Y_{P/S}) was higher for the culture with pyridoxine, 21.84 U/g, than the culture without pyridoxine, 13.95 U/g.

TABLE 2
Factors obtained of productivity experiment. Total substrate =
C_{glucose} + Cₘₐₗₜₒₛₑ/0.947. Y_{P/X} = protein to dry
cell mass yield, Y_{P/S} = protein to substrate yield.

| Factors | w/o pyridoxine | w/pyridoxine |
|---|---|---|
| Initial maltose [g/L] | 47.62 | 47.62 |
| Initial glucose [g/L] | 10.00 | 10.00 |
| Initial total substrate [g/L] | 60.28 | 60.28 |
| Final maltose after 24 h [g/L] | 7.68 ± 1.30 | 6.02 ± 0.993 |
| Final glucose after 24 h [g/L] | 8.56 ± 1.90 | 4.92 ± 0.397 |
| Final total substrate after 24 h[g/L] | 16.67 | 11.28 ± 1.40 |
| XynB concentration after 24 h [U/ml] | 17.32 ± 3.15 | 11.27 ± 4.38 |
| Dry cell mass [g] | 0.68 ± 0.13 | 0.78 ± 0.08 |
| Y_{P/X} [U/g] | 507.42 ± 37.54 | 194.21 ± 52.81 |
| Y_{P/S} [U/g] | 21.84 ± 8.57 | 13.95 ± 5.64 |
| Productivity [U/g · h] | 21.14 ± 1.56 | 8.09 ± 2.20 |

### CONCLUSION

It was successfully demonstrated that a fungus mutant requiring pyridoxine for growth can maintain enzyme production even when growth is limited by limiting pyridoxine. However, continuous enzyme production cannot go on forever. Pyridoxine needs to be replenished frequently. Since pH correlates with pyridoxine depletion, monitoring the pH allows identifying the correct moment when pyridoxine needs to be added.

Pyridoxine limitation causes stress on the cell, which induces conidiation and melanin formation. The increased stress on the cells due to pyridoxine limitation was observed to have no effect on XynB formation. With a decrease in protein formation, the activity increases, which keeps the volumetric activity relatively constant.

During transplanting the medium becomes darker in color over time. It is assumed that the darker coloration is due to an elevated melanin formation. An increase in melanin formation was reported when filamentous fungi initiate conidiation (Jørgensen et al. 2010). Conidiation is usually a response to energy or carbon starvation to conserve the cell until it is transported to a nutritional environment. Stress induces the transcription of brlA (Skromne et al. 1995) and the transcription factor brlA is a positive regulator for conidiation (Adams and Timberlake 1990). At the moment when all pyridoxine was depleted, the protein production and the PH were at a minimum and the medium became a light yellow. This shows that no melanin, and therefore, no conidiation took place. However, when the same culture was placed again on medium with pyridoxine, the medium became darker again and melanin was formed.

### Example 2

### Large Scale Production in a Trickle Bed Reactor (TBR)

To enhance enzyme productivity, an exemplary trickle bed reactor (TBR) was designed. In such a reactor, the macrostructure of the cultured fungus is maintained by keeping the mass transfer at an optimum. The support that is used is inert and the substrates for enzyme expression and growth are in a dissolved form. Furthermore, the system allows for maintaining a large cell mass with a large surface area. The reactor system is modeled for continuous enzyme production. XynB production with the reactor was compared to shaking flask and static tray fermentation methods, in order to simulate SmF and SSF. Further, XynB production in the reactor was tested with and without growth limitation induced by pyridoxine limitation.

### Materials and Methods

### Culture Medium

The medium was composed of 50.0 ml/L 20× Clutterbuck salts (120 g/L NaNO₃, 10.4 g/L KCl, 10.4 g/L MgSO₄, 30.4 g/L KH₂PO₄), 1.0 ml/L 1000× trace elements (22 g/L ZnSO₄.7H₂O, 11 g/L H₃BO₃, 5.0 g/L MnCl₂.7H₂O, 5.0 g/L FeSO₄.7H₂O, 1.6 g/L CoCl₂.5H₂O, 1.6 g/L CuSO₄.5H₂O, 1.1 g/L Na₂MoO₄.4H₂O, 50 g/L Na_{z}-EDTA), 10.0 g/L glucose monohydrate, 50.0 g/L maltose monohydrate and 1 ml/L of a 1 g/L pyridoxine solution. For the pyridoxine limitation test, the medium did not contain pyridoxine. The pH was adjusted to 6.5 using 10 N NaOH. The medium was sterilized by autoclaving at 121° C. for 20 min.

### Reactor Design

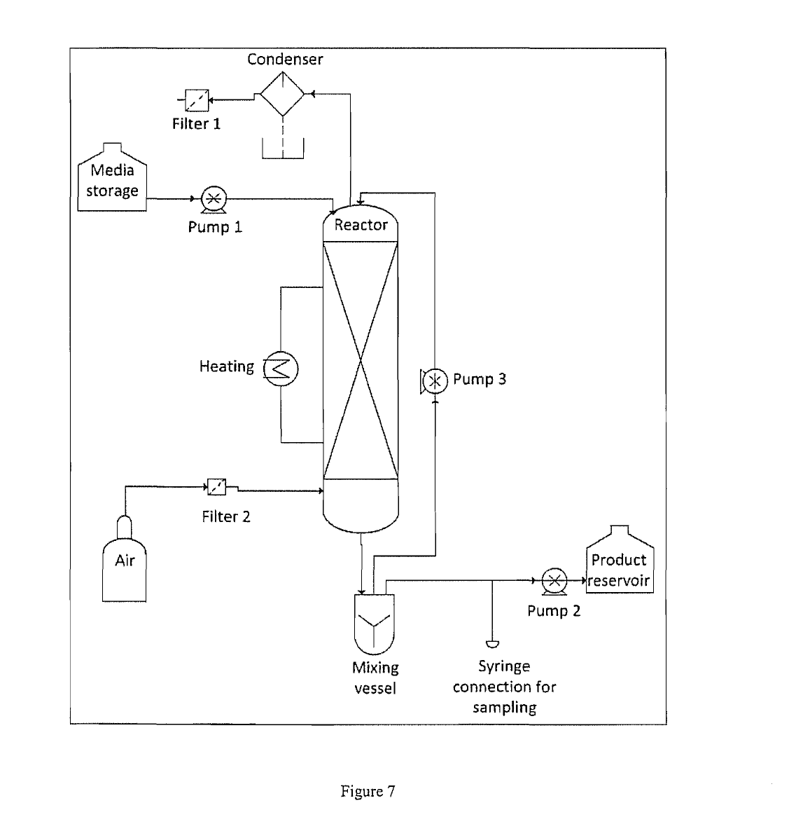

FIG. 7 shows a schematic of an exemplary TBR design. The reactor is composed of a glass column with an inside diameter of 10.5 cm. The height is 60 cm. The packing consists of lava rocks purchased from a local hardware store, usually used for outside grills. The diameter of the rocks varied between 3 and 4 cm. The inlets for fresh and recycled medium were placed on top of the reactor. On the bottom a stainless funnel led the medium into a 250 ml baffled flask (“mixing vessel” in FIG. 7). The baffled flask was placed on a magnetic stirrer. The whole reactor was able to fit into an autoclave for sterilization. A heating tape was wrapped around the column to keep a constant temperature of 37° C. Three peristaltic pumps (MasterFlex 7523-20, Barnant Co., Barrington, Ill., USA) were used for medium flow. The influent and effluent flow rates varied from 0.5 to 1.0 ml/min. One pump was used to pump in fresh medium into the TBR. The second pump took medium out from the baffled flask at the same rate as the first pump. The third pump was used to pump medium from the baffled flask back into the reactor. The recycling of medium at various rates (224, 112, and 56 m/min) allowed higher substrate utilization, mixing and enhanced enzyme productivity. The tubing that connected the baffled flask with the product reservoir had an attachment that allowed sampling. Air was pumped into the reactor at 1.15 L/min (standard temperature and pressure) at the bottom through a 0.2 μm sterile filter and exited the column at the top.

### Reactor Model

Several models are available to design reactors based on unicellular organisms. However, for filamentous fungi applied in a TBR, no model has been established. Thus, several parameters were determined experimentally. The specific and maximum growth rates as well as productivity of A. nidulans were 0.061 h⁻¹ (for 59.3 g/l total substrate concentration), 0.311 h⁻¹, and 21.14 U/g·h, respectively.

The following assumptions were made in the development of this embodiment of the model:
The fungus will have the same yields (Y_{X/S}, Y_{P/S}, and Y_{P/X}) in the reactor as those observed with Petri dish experiments for the same initial substrate concentration.
The specific growth rate found in the Petri dishes is equal to the specific growth rate in the reactor.
The total available medium inside the reactor includes medium to saturate the lava rocks.
Maltose and glucose are utilized at equal rates.
A high recycle flow rate allowed for the assumption that the reactor behaved like a continuously stirred tank reactor (CSTR).

Since it was not usually possible to measure the exact cell mass inside the reactor, a theoretical cell mass based on Y_{X/S} was calculated. Assuming complete substrate utilization during the growth phase, the cell total mass (X) could be calculated:

X=Y_{X/S}·S_{T}

with Y_{X/S}=cell mass to substrate yield and S_{T}=Total substrate mass [g] in the reactor. The substrate uptake rate R [g/h] could now be calculated using the specific substrate uptake rate and the total cell mass:

R=qs·X

with qs=specific substrate uptake rate in g substrate/g cells*h.

Using the active volume, a volumetric substrate uptake rate R_{V} [g/L*h] can be calculated:

R_{V}=R/Vₐ

with Vₐ=active volume [L].

The flow rate Q was calculated using the volumetric substrate uptake rate and the substrate concentration in the inlet:

Q=R_{V}/S

with S=substrate concentration [g/L]. This flow rate was determined based on the assumption that all substrates are consumed according to the rates and yields determined in experiments done in Petri dishes.

### Reactor Process

The primary target of this investigation was to achieve a continuous system for enzyme production with μₙₑₜ equal to zero. The reactor process was divided into two steps. The first step was the startup and growth of cell mass. The second step initiated the growth limitation with continuous product formation. This step required a change to medium without pyridoxine.

In the first step, 600 ml of medium were pumped into the reactor and recycled until all packing material was saturated with liquid. Twenty-five ml of a concentrated spore solution (8×10⁷ spores/ml) containing 20.0 mg spores of A. nidulans (dry weight) was added with a syringe on top of the reactor. The remaining free medium, about 60 ml, were then continuously recycled at 224 ml/min without the addition or extraction of medium, until all substrates were depleted and the fungus culture was grown.

In the second step, the free medium was extracted from the reactor, and replaced with medium that had no pyridoxine. A constant flow of pyridoxine-free fresh medium was pumped into the reactor. At the same rate, medium was extracted from the reactor and collected. The medium in the mixing vessel, which had the same composition as the effluent, was frequently monitored for maltose, glucose and protein concentration analysis. The pH was also monitored frequently. When the pH started to decrease to 6.5, indicating that pyridoxine was exhausted, a 0.5 ml aliquot of a 1 g/L pyridoxine solution was added into the reactor. During step 2, the medium was recycled at various high flow rates (224, 112, 56 ml/min). This allowed the assumption that at any moment and location, the substrate concentration throughout the reactor was equal.

### Analysis of Flow Rate Through Reactor

In this embodiment the glass column was filled with a known mass of air dried lava rocks. The height of the packing material was 50 cm with a column diameter of 10.5 cm. Before the flow rate could be measured, the tubing of the system was filled with DI water. A graduated cylinder was filled with 1.0 L of DI water. At various flow rates the remaining liquid in the graduated cylinder was recorded after the system reached steady state. At zero flow rate the mass of water attached to the lava rocks (holding capacity) could be obtained at steady state. The holding capacity was also measured in a different test, where rocks were first submerged in water and then weighed after all the surface water was drained.

### Enzyme Production in Static Tray

A stainless steel tray was used for culturing. The tray was filled with 500 ml medium, containing a minimal medium with 50.0 ml/L 20× Clutterbuck salts (120 g/L NaNO₃, 10.4 g/L KCl, 10.4 g/L MgSO₄, 30.4 g/L KH₂PO₄), 1.0 ml/L 1000× trace elements (22 g/L ZnSO₄.7H₂O, 11 g/L H₃BO₃, 5.0 g/L MnCl₂.7H₂O, 5.0 g/L FeSO₄.7H₂O, 1.6 g/L CoCl₂.5H₂O, 1.6 g/L CuSO₄.5H₂O, 1.1 g/L Na₂MoO₄.4H₂O, 50 g/L Na_{z}-EDTA), 10.0 g/L glucose, 50.0 g/L maltose and 1 ml/L of 1 g/L pyridoxine solution with an adjusted pH of 6.5. Spores of A. nidulans with a pyridoxine marker cultured on three agar plates containing minimal medium were manually scratched off the surface and added to the medium in the tray under sterile conditions. The trays were incubated at 37° C. for 48 h. Since the fungus grows on top of the liquid, a separation of liquid from fungus was achieved by filtration through a filter paper vacuum filtration unit. After filtration the enzyme concentration was determined.

### Comparison of Tray and Shaking Flask Enzyme Production

The purpose of this experiment was to determine if the protein production was higher using a tray in comparison to using a flask. Shaking was shown in the literature to alter the growth form of filamentous fungi to pellets, while a surface grown fungi develops a mycelium with hyphae directed to the source of nutrients. The same medium volume of 180 ml was added to a tray and a 500 ml Erlenmeyer flask. Both cultures were inoculated with spores to a final concentration of 4×10⁵ spores/ml and grown at 50° C. The flask culture was agitated at 225 rpm. Samples were taken at 0, 24, 48, 72 and 96 h and analyzed for protein, glucose and maltose concentration.

In order to compare the enzyme production between the tray, flask and reactor operation, the total mass protein over a fixed period of time was calculated and standardized with dry cell mass. For the reactor the total dry cell mass was determined after the run was completed. The protein formation over a steady state period of time with a known flow rate was used to determine total protein produced within that time period. The following equation was used for this embodiment of the reactor:

$${P}_{reactor} = \frac{{\overline{p}}_{{T}_{2} - {T}_{1}} \cdot Q \cdot ( {t}_{2} - {t}_{1} )}{X}$$

With P_{Reactor}=total protein mass per dry cell mass [g/g], Pₜ₂₋ₜ₁=average protein concentration [g/L] during the time period between t₁ and t₂ [h], Q=flow rate of continuous operation [L/h] and X=dry cell mass [g].

The equation below is used to calculate the total protein per cell mass from both, tray and flask cultures. These systems are batch fermentations, which simplifies the equation.

$$P = \frac{\overline{p} \cdot V}{X}$$

With P=total protein per dry cell mass (P_{F} and P_{T} for flask and tray, respectively) [g/g], p=protein concentration [g/L], V=liquid volume [L] and X=dry cell mass.

The dry cell mass is obtained by filtering the medium and cells through a filter paper at the end of the experiment and drying overnight at 105° C.

### Determination of Protein Concentration

The protein concentration was determined using a Bradford assay. Forty μl of Bradford commassie solution were pipetted into a well of a 96-well plate. The amount of enzyme solution depended on the protein concentration received. A typical amount is 40 μl. DI water was added to achieve a total volume of 200 μl. The blank well contained only 40 μl Bradford solution and 160 μl DI water. The absorbance was measured using a UV-Vis 96-well plate reader (Tecan Infinite M200, Männedorf, Switzerland) at 595 nm.

### Determination of XynB Activity

Xylanase B activity was measured on xylan from beechwood (Fisher Scientific, Pittsburgh, Pa.). Forty-eight μl of 1.0% substrate solution and 2 μl of enzyme solution were added into a 96-well plate. The plate was inoculated in a water bath at 50° C. for 7 min. After inoculation, the 96-well plate was removed from the water bath and 50 μA of dinitrosalicylic acid (DNS) reagent were added immediately to terminate enzymatic hydrolysis. The 96-well plate was placed in a second water bath at a temperature of 100° C. for 5 min to achieve the color formation. After the reaction time, 100 μl of the liquid was transferred in a 96-well reading plate and analyzed at 575 nm for reducing sugar concentration. With the following equation, the specific activity was calculated based on the spectrophotometer values.

$$U = ( ( ( A - 0.047 ) / F ) * ( \frac{{V}_{assay}}{t } ) ) / {p}_{enzyme}$$

With U=specific activity [μmol of reducing sugar/mg protein·min], A=absorbance at 575 nm, F=calibration factor, V_{assay}=assay volume (200 μL), t=incubation time [min] and p_{enzyme}=mass protein in enzyme used [mg].

The final results are given in XynB activity units per volume, U_{V}.

U_{V}=U·C_{P}

With C_{P}=protein concentration in mg/ml.
Protein Analysis with SDS-PAGE

Additional characterization of the proteins was done by sodium dodecyl sulfate polyacrylamide gel electrophoresis (SDS-PAGE). A 12% separation gel was made by mixing 1.7 ml H₂O, 1.25 ml 1.5 M Tris, 50 μl 10% SDS, 2.0 ml 30% acryl, 25 μl 10% ammonium per sulfite (APS) and 25 μl tetramethylethylendiamin (TEMED). The stacking gel was composed of 2.5 ml H₂O, 0.38 ml 0.5 M Tris, 0.03 ml 10% SDS, 0.5 ml 30% acryl, 0.03 ml APS and 3.0 μL TEMED. Samples were concentrated using ultrafiltration (Stirred Ultrafiltration Cell Model 8400, Millipore, Billerica, Mass., USA). An aliquot equivalent to 10 μg protein was mixed with 15 μl of 2× Laemmli buffer (4% SDS, 20% glycerol, 10% 2-mercaptoethanol, 0.004% bromphenol blue, 0.125 M Tris HCl) and boiled for 5 min for optimal band resolution. The solution was then injected into the gel. After completion of the SDS-PAGE run, the gels were stained with Bio-Safe™ Coomassie Stain (Bio-Rad, Hercules, Calif., USA) and de-stained with water.

### Sugar and Organic Acid Analysis

Concentrations of glucose, maltose and urea were analyzed on an HPX-87P column (Bio-Rad, Sunnyvale, Calif.). The eluent was HPLC grade DI-water with a flow of 0.6 ml/min at 85° C. and a refractive index detector (1100 Series Agilent, Santa Clara, Calif., USA) (Sluiter et al. 2008c). Organic acids were analyzed on a HPX-87H column (Bio-Rad, Sunnyvale, Calif.). The eluent was 0.1 N sulfuric acid with a flow rate of 0.6 ml/min at 60° C. A refractive detector (1100 Series Agilent, Santa Clara, Calif., USA) was used for detection (Sluiter et al. 2008c).

### Calculation of Yields

In order to compare the different XynB productions methods used in this study, in an embodiment a common base needs to be established. Cell mass and liquid volume may differ, which can lead to false conclusions if just XynB concentration is compared. Since typical enzyme activity is given in U (μmol/min), all yields are based on the volumetric XynB activity, U_{V}, in U/L. Substrate and cell mass concentrations are given in g/L. The product from substrate yields, Y_{P/S}, is calculated for the tray and flask experiment as:

$${Y}_{P / S} = \frac{U / V}{( {S}_{0} - {S}_{96} )} [ U / g ]$$

With U=XynB activity in U, V=volume of fermentation broth after 96 h in L, S₀=initial substrate concentration in g/L and S₉₆=substrate concentration at 96 h in g/L. Similarly can the product from substrate yield, Y_{P/X}, be calculated:

$${Y}_{P / X} = \frac{U / V}{{X}_{96}} [ U / g ]$$

With X₉₆=cell mass concentration after 96 h in g/L. The initial cell mass can be neglected. The cell mass from substrate yield (Y_{X/S}) was calculated with following equation:

$${Y}_{X / S} = \frac{{X}_{96}}{( {S}_{0} - {S}_{96} )} [ g / g ]$$

### Results and Discussion

### Comparison of Enzyme Production in Static Tray and Shaking Flask

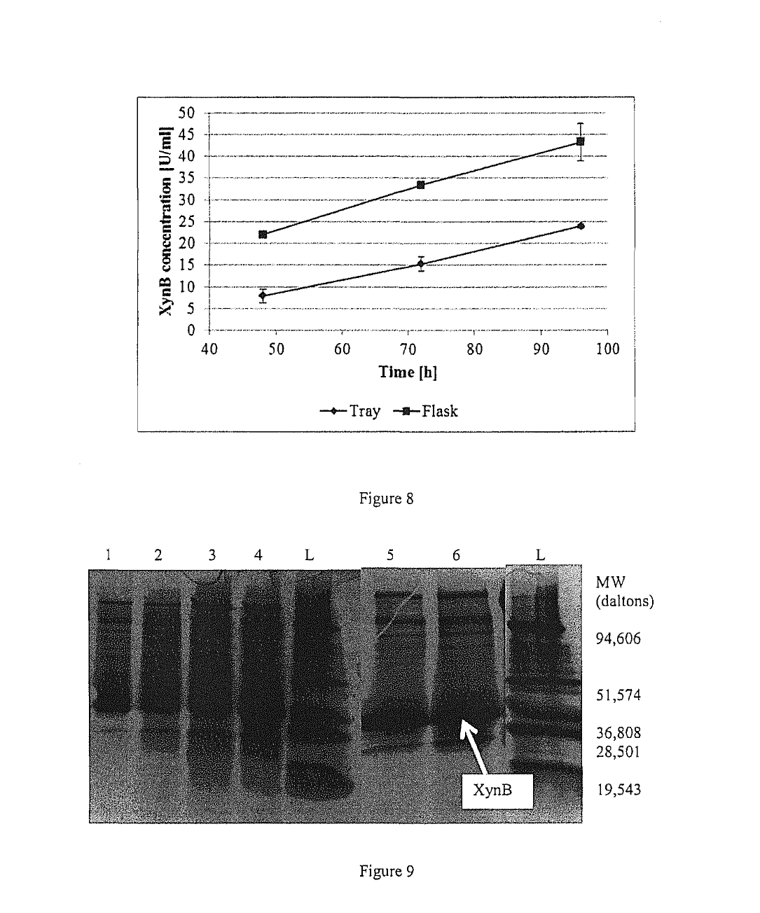

One purpose of this study was to show that a culture grown as a static tray culture (simulating a SSF) would produce higher titers of the client enzyme compared to a shaking flask by using the same liquid volume and spore inoculation. As it can be seen in FIG. 8, the XynB concentration over time was about two times higher with the shaking flask culture than with the static tray after 96 h. The SDS-PAGE analysis revealed that the tray shows larger bands of XynB compared to the flask, indicating a higher purity of XynB in the total protein mix (FIG. 9). This observation confirms the results found in previous studies comparing SSF with SmF (Viniegra-González et al. 2003), where SSF fermentations led to higher enzyme purity. However, a comparison of the yields shows that the shaking flask produces XynB more efficiently than the static tray. The product to substrate yield (Y_{P/S}) was 780.72 U/g for flask fermentation and 543.16 U/g for tray fermentation after 96 h. The flask culture also resulted in a greater product from cell mass yield, Y_{P/X}=1849.77 U/g, compared to the tray experiment with Y_{P/X}=480.05 U/g. Due to the high cell mass of 50.0 g/L produced in the static tray, the cell mass from substrate yield (Y_{X/S}) is higher, 1.13 g/g, compared to the flask, 0.42 g/g.

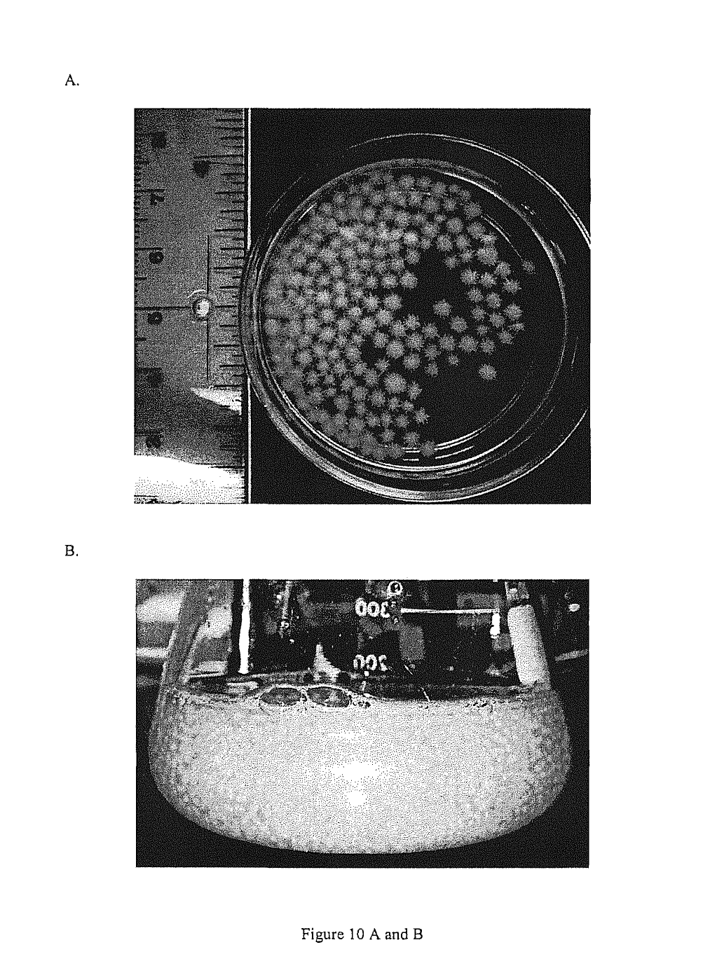

The fungal morphology was very different in both cultures. While the static tray showed a dense mycelium on top of the surface, the shaking culture produced pellets (FIG. 10A) with a diameter of about 2-3 mm. It has been shown that the pellet size depends on the inoculum concentration. With increasing inoculum spore concentration, the pellet size decreases (\check{Z}nidar\check{s}i\check{c} et al. 2000). Other factors affecting the pellet formation are shear stress (Fujita et al. 1994) and pH (\check{Z}nidar\check{s}i\check{c} et al. 2000). The pellet size was found to affect the protein formation, where a decrease in pellet size causes an increased product formation (El-Enshasy et al. 1999). However, in order to extract the desired protein from the fermentation broth after fermentation, the fungal mass has to be separated, which is more difficult with the pellet culture than with the static tray culture. The density of the pellets is similar to the density of the fermentation broth (FIG. 10B). Settling or centrifugation are less effective in separating the pellets while filtering requires a high energy demand due to the viscosity of the broth. This was also observed when the samples were filtered through 0.2 μm syringe filters for sample analysis. A relatively large amount of force was required to press the liquid through the filter. In this process the pellets needed to be disrupted to extract the liquid with the syringe. It is believed that downstream processing of a fungal pellet solution is more energy and process intensive compared to the fermentation broth from a static culture.

### Flow Rate Through Reactor

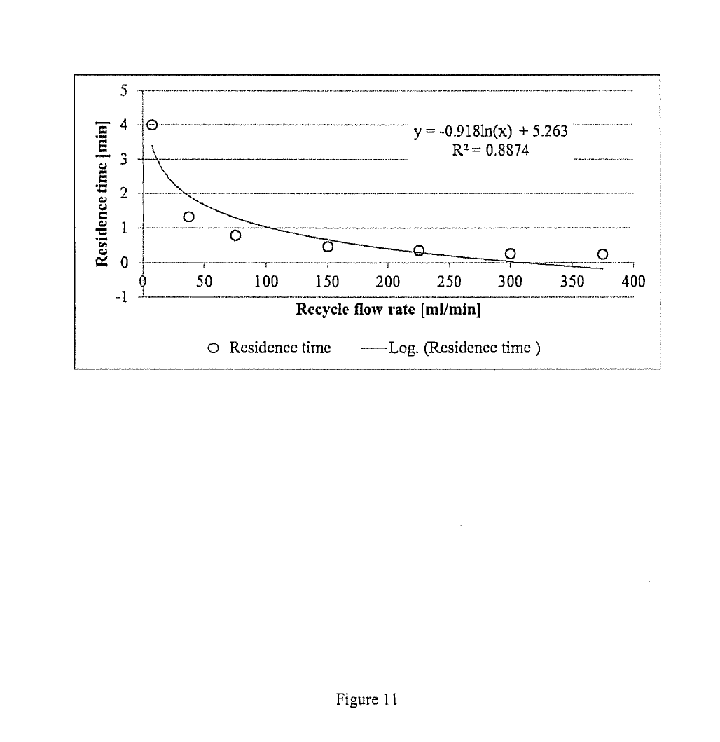

The mass of rocks used for this experiment was 2,501.6 g. The packing was 50 cm high with a diameter of 10.5 cm. The rocks were air dried prior to the experiment and the tubing was filled with water. FIG. 11 shows the residence time over flow rate. A logarithmic relationship can be seen. Between each change of flow rate, the system was allowed to reach steady state for 30 min. It can also be seen that when no flow was applied, 560 ml of water stayed in the column. This amount of water is adsorbed to the rock surface and in capillaries within the rocks. In a separate experiment the water holding capacity was determined on a mass of water per mass of lava rocks basis. By submerging a known mass of rocks in water and weighing the rocks after draining the surface water a factor of 0.214 g water/g rocks was calculated. When this factor is applied to the mass of rocks used in this experiment the amount of water adsorbed to the rocks would be 535 g. The volume per mass ratio of the rocks was determined by a volume replacement experiment. The factor is 0.579 ml/g. Using this factor the static liquid holdup in the rocks was 0.370 ml water/ml rocks.

### Enzyme Production in Reactor

Enzyme Production without Pyridoxine Limitation

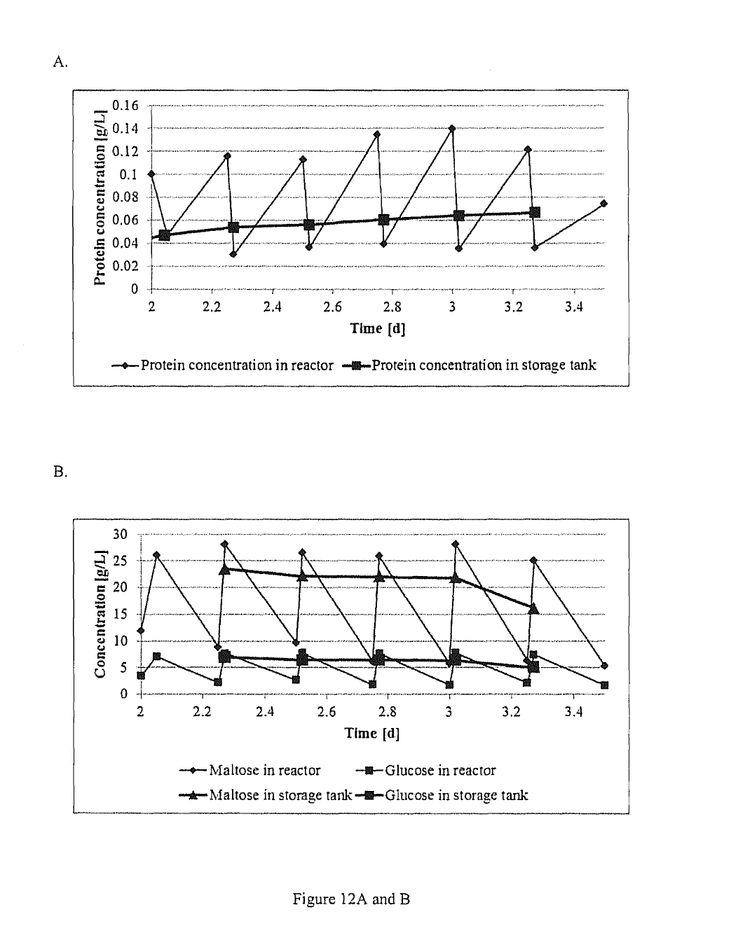

The reactor design was tested with and without pyridoxine limitation. After 42 h of inoculation, a uniform fungus formation was observed, which was when semi continuous operation was initiated. The medium was recycled with flow rates equal or higher than 51 ml/min to achieve a uniform substrate concentration throughout the column. Every 6 h a volume of 240 ml was taken out from the mixing vessel into the storage tank and replaced with fresh medium. FIG. 12A shows the protein concentration in the reactor, as well as in the storage tank.

Also shown in FIG. 12B is the glucose and maltose concentration. Between day 2 and 3.3, a constant enzyme production was achieved. However, at day 3.5, the tubing in- and outlets were clogged and eventually the process was terminated. The zigzag pattern in protein concentration was due to the semi continuous operation. Right before the feeding, the concentration of protein was highest in the reactor. After feeding the reactor with fresh medium, the protein concentration was low due to a dilution with fresh medium. The protein concentration in the storage tank slightly increased from 0.047 g/L at day 2 to 0.067 g/L after 3.3 days. During the 6-h-periods, 80% of glucose and 77% of maltose were utilized. However, as can be seen in FIG. 12B, the maltose and glucose concentration in the storage tank was higher. Including the storage container, a maltose utilization of 53% and a glucose utilization of 35% was achieved. The activity was not measured during the reactor run. Using an average activity per protein concentration of 575 U/g protein, obtained from later experiments, an estimated average XynB production of 31 U/ml was produced and present in the storage tank. While this test demonstrated a possible continuous enzyme production with a trickle bed reactor, the high risk of clogging needed to be addressed in order for the reactor to be operational over a prolonged time.

Enzyme Production with Pyridoxine Limitation

A continuous enzyme production with limited risk of clogging was achieved by reducing growth with pyridoxine limitation. In a petri dish experiment, the feasibility to produce protein continuously without fungal growth was demonstrated. This concept was applied to the reactor design.

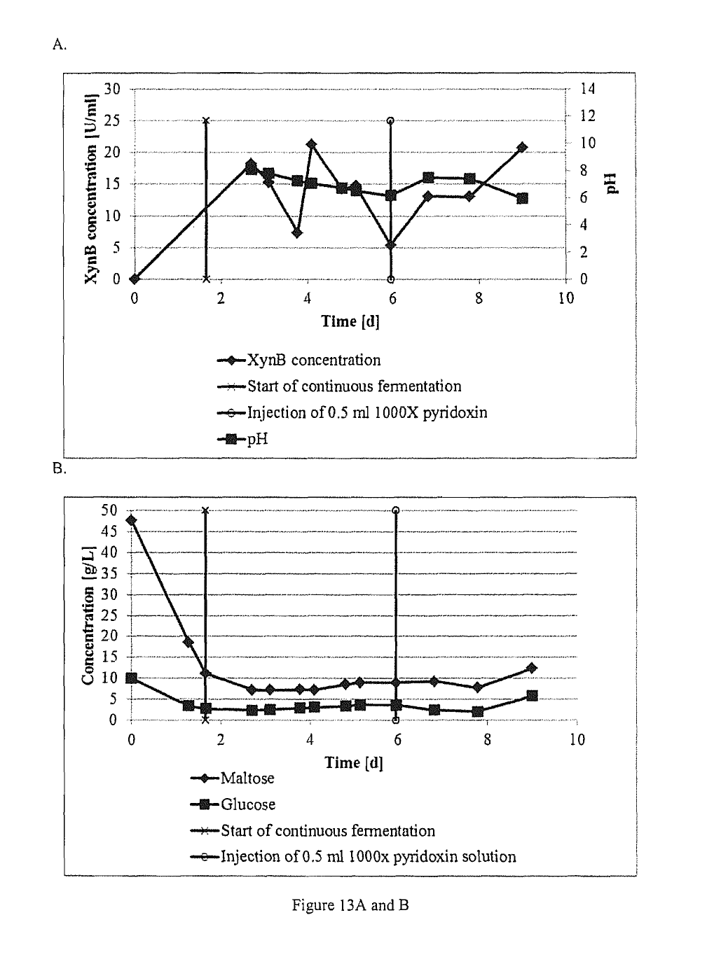

In a first embodiment, the reactor was filled with 2,502 g of lava rocks. The water holding capacity of the lava rocks was previously determined. A volume of 550 ml of medium was required to saturate 2,502 g of rocks. After autoclaving the reactor was filled with 700 ml of medium with pyridoxine, which was recycled at a flow rate of 224 mL/min until all rock surfaces were wetted and a steady level of 160 ml of medium in the mixing vessel was achieved. A fungal spores suspension (8×10⁷ spores/ml) containing 20.0 mg spores (dry weight) was injected and allowed to grow for 1.7 days. After 1.7 days the medium inside the reactor was allowed to trickle down into the mixing vessel and then was pumped out. The same amount of fresh medium without pyridoxine (150 ml) was added and recycled at a flow rate of 224 ml/min until the desired level of medium (160 ml) in the mixing vessel was reached. Then the system was switched to continuous operation with an in- and effluent outflow rate of 1.0 ml/min (D=0.0014 h⁻¹). After 2.1 days of inoculation, a XynB activity of 14.4 U/ml was reached, which was held for 4 days (FIG. 13A). As expected the pH dropped from 8.08 at 2.7 d to 6.18 at 6.0 d. The same behavior was observed during a petri dish experiment and is an indicator of when pyridoxine needs to be replenished. At 6.0 d, 0.5 ml of 1000× pyridoxine solution was added. As can be seen in FIG. 13A, the XynB activation increased again to 13.1 U/ml and the pH increased to 7.49.

The substrate concentration can be seen in FIG. 13B. After continuous production was initiated, the concentration of maltose was 7.3 g/L, which is equivalent to a substrate utilization rate of 85%. The concentration of glucose was 3.0 g/L, which is equivalent to a substrate utilization rate of 75%.

Due to contamination, the continuous product formation stopped after 7.8 days. However, the process was allowed to continue while an attempt to remove the contamination was tried by the addition of ampicillin. During this period it was observed that the protein concentration dropped to a minimum of 0.017 g/L after 9 days. The activity measured was 1194.79 U/mg. This would result in a volumetric activity of 20.3 U/ml. Even with reduced protein production, the XynB metabolism was unaffected.

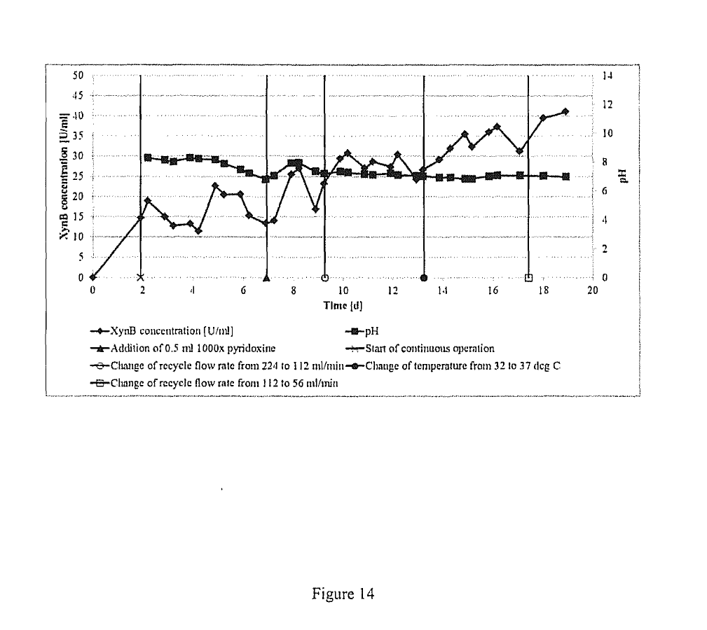

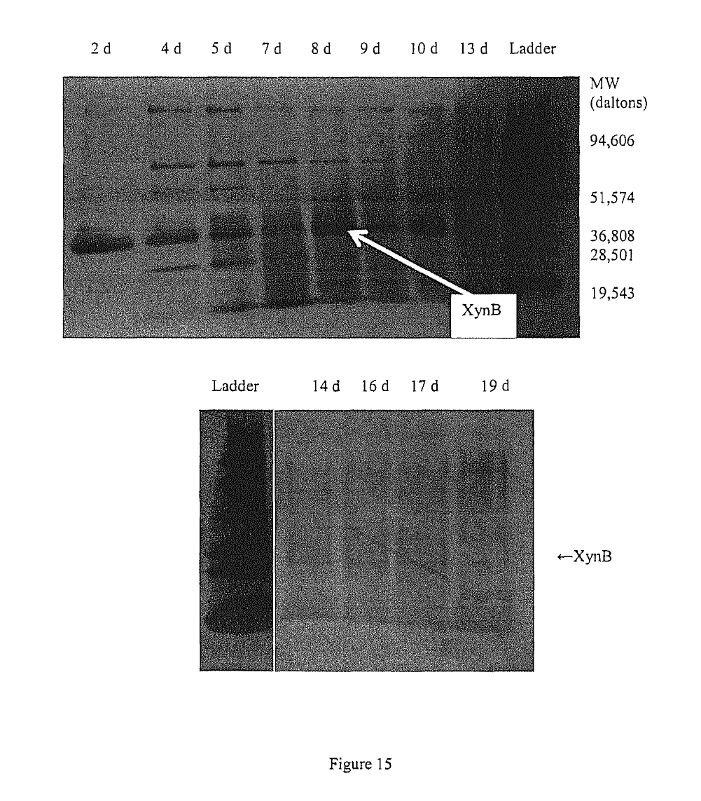

In a second embodiment, the fermentation conditions were modified to achieve an improved XynB production. The lava rocks from the previous experiment were replaced with a fresh set with a total mass of 2,353 g. The reactor was filled with 740 ml pyridoxine containing medium and recycled to achieve a uniform wetting of the lava rocks and a steady liquid level in the mixing vessel (160 ml). The same amount of spores was added as in the previous experiment (8×10⁷ spores/ml). After 1.9 days continuous operation was started with an influent and effluent flow rate of 0.5 ml/min (D=0.0007 h⁻¹). Table 3 shows an overview of the most important results. A continuous XynB production was achieved for 7 days with a concentration of 17.81 U/ml (FIG. 14). After day 6.9 0.5 ml of 1000× pyridoxine solution were added since a decrease in XynB and pH was observed. At day 9.3 the recycle flow rate was reduced by 50% to 112 ml/min. The change of flow rate resulted in an increase of XynB production to an average of 28.09 U/ml and increased the productivity from 3.6 U/g cells*h to 5.7 U/g cells*h. A decrease of recycle flow rate of 50% doubles the residence time. The medium is longer in contact with the mycelium, which explains the increase in XynB concentration. Further improvement of XynB production was achieved when the temperature was increased from 32° C. to 37° C. at day 13.3. The XynB concentration increased to 33.36 U/ml and productivity increased to 6.7 U/g cells*h. The improvement by adjusting the temperature to 37° C. was expected since 37° C. is the optimum growth temperature for A. nidulans. At day 17.4 the flow rate was again reduced by 50% to 56 ml/min, which increased of XynB production to 40.26 U/ml and a productivity of 8.1 U/g cells*h. Further confirmation of XynB production was given by SDS-PAGE analysis. FIG. 15 shows the XynB bands for selected time points. The XynB bands are the most predominant bands in the pictures and indicate a high purity of XynB.

TABLE 3
Comparison of product yields at different
time intervals with 0.5 ml/min flow rate.

| | Average | | | | |
|---|---|---|---|---|---|
| | product | Average sugar | | | |
| Time | formation | concentration | Dry cell | Y_{P/S} | Y_{P/X} |
| [d] | [U/ml] | [g/L] | mass [g] | [U/g] | [U/g] |
| 2-9 | 17.81 | 21.62 | n.a. | 494.78 | — |
| 9-13 | 28.09 | 16.23 | n.a. | 678.56 | — |
| 13-17 | 33.36 | 7.48 | n.a. | 665.25 | — |
| 18-19 | 40.26 | 4.34 | 148.8 | 755.58 | 626.08 |

In this embodiment the reactor produced high titers of XynB. A comparison of XynB with SSF xylanase production in the literature is not directly possible since the present study is a continuous process; whereas; SSF is performed in batch mode. Typical xylanase production results from SSF are given in U/g of solid substrate, while this study uses U/ml. Azin et al. (2007) reported a xylanase production of 600 U/g substrate using Trichoderma longibrachiatum on a mixture of wheat bran and wheat straw. Converting the xylanase concentration to U/ml by incorporating the substrate mass and volume liquid used for enzyme extraction results in a volumetric activity of 35.3 U/ml. Similarly, the xylanase production achieved by Gessesse and Mamo (1999) was reported to be 600 U/g using an alkaliphilic Bacillus sp. on wheat bran. Conversion to volumetric activity gives 0.6 U/ml. Park et al. (2002) reported an about 10-fold higher xylanase production of 5,000 U/g substrate compared to other literature findings. The researchers used A. niger on rice straw. When substrate mass and extraction liquid volume are incorporated to the result, the volumetric activity was 125 U/ml. The difference with other findings may be explained by the usage of a different organism.

The current embodiment of the reactor design also produced higher XynB concentrations (FIG. 15) compared to the static tray culture and reached the level of the shaking flask culture. Product from cell mass and substrate yield were both higher for the reactor compared to the static tray. The reactor achieved an Y_{P/X} of 626.1 U/g and an Y_{P/S} of 755.7 U/g (Table 3). By recycling the medium and increasing the surface for mycelium growth, the reactor likely allows improved mass transfer over the static tray.

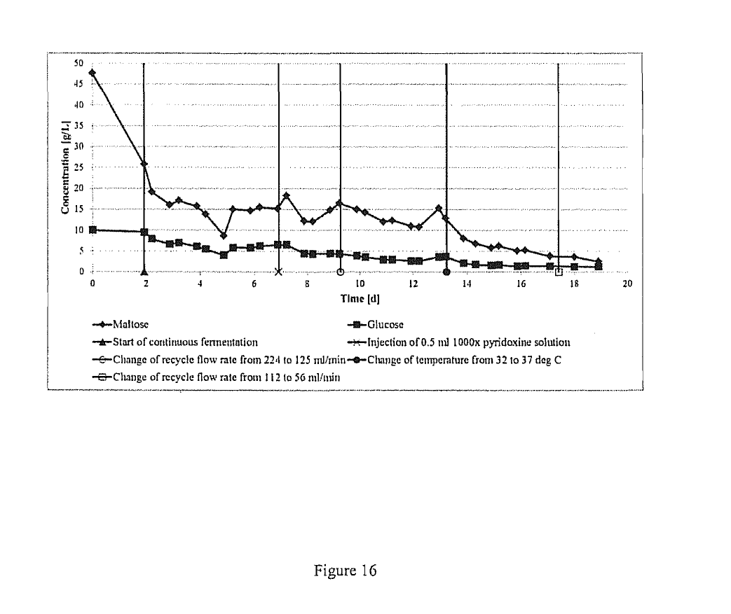

The substrate consumption throughout the reactor run increased with changes in operation conditions. The highest maltose and glucose utilization was achieved at day 18 with the lowest recycle flow rate (56 ml/min) and 37° C. (FIG. 16). During the highest product formation, 92% and 78% of maltose and glucose were consumed, respectively. With a lower recycle flow rate, the residence time of flowing liquid through the packing increased, which allowed for improved diffusion of substrate into the fungus matrix. However, the mass transfer in the reactor can be further improved. Channeling occurred due to insufficient medium distribution on top of the column. The channeling led to areas in the bed where no medium or only limit amounts of medium came into contact with the mycelium. These issues could be addressed in a number of ways, including without limitation by changing the diameter to height ratio of column and with an improved medium inlet on top of the column, such as a spray nozzle.

### Prediction of XynB Production and Substrate Utilization

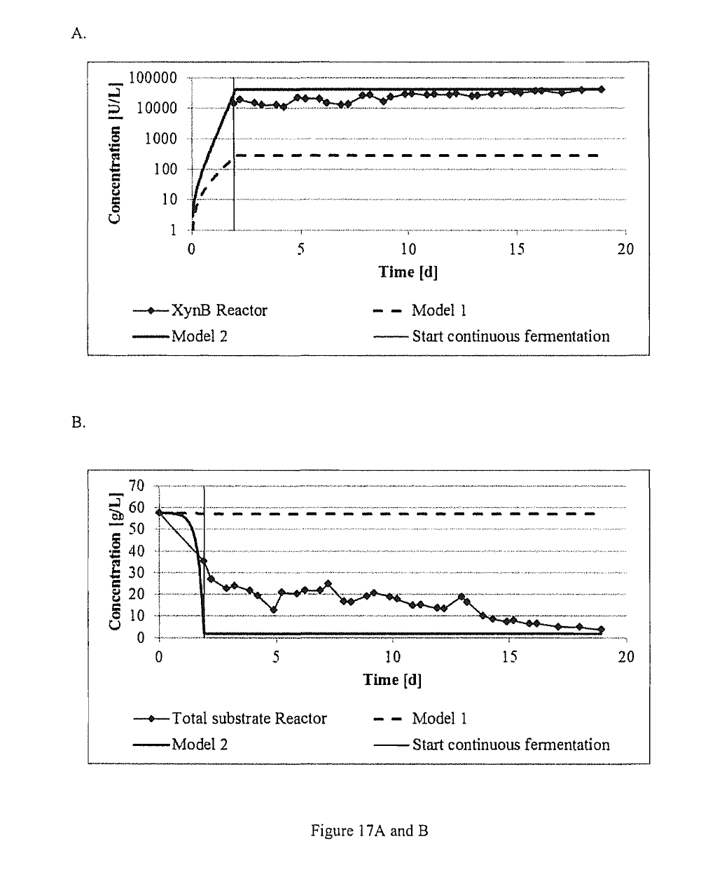

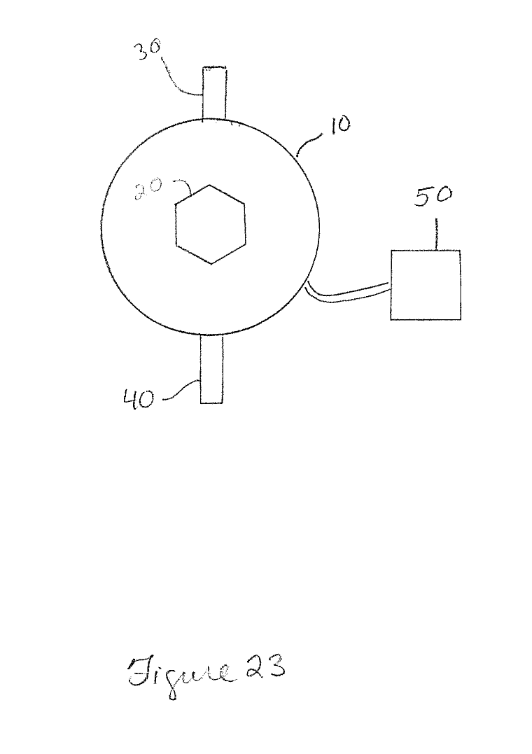

The XynB production and substrate utilization were modeled using CSTR model equations. CSTR modeling compared to packed-bed reactor modeling leads to a more simplified prediction of product formation. In this experiment the recycle flow rate was high enough, with a residence time of 22 s, to assume a constant concentration of substrates, product and cell mass throughout the reactor column and the mixing vessel. It was not possible to measure cell mass concentration during the experiment. With a known initial cell mass concentration, the cell mass could be predicted over time. Using this predicted cell mass, the XynB formation and the substrate utilization could be predicted. The required parameters were, Sₒ, which was maltose (47.6 g/L), glucose (10 g/L), or the summation of both (57.6 g/L), X₀=0.033 g/L, μₙₑₜ=0.061 h⁻¹, Y_{P/X}=480 U/g, Y_{X/S}, which was different for maltose (1.2), glucose (19.2), and both together (1.13). Model 1 is based on summation of both sugars as substrate concentration. FIG. 17A shows the actual XynB production in the reactor and the predicted XynB production (Model 1). It also shows a different prediction, Model 2, which was calculated with an adjusted net growth rate. The adjusted growth rate was back calculated using the actual XynB production at day 18 in the reactor. With the known product concentration at day 18 and Y_{P/X}, the theoretical cell mass concentration can be determined. With the theoretical cell mass concentration and the known initial cell mass, the μₙₑₜ necessary to achieve the theoretical cell mass at 48 h can be calculated. As can be seen in FIG. 17B, which shows the total substrate utilization, Model 2 is more suitable. Various graphs predicting cell growth, maltose and glucose utilizationare shown in FIGS. 21 and 22A and B. The adjusted net growth rate was 0.164 h⁻¹. The previous μₙₑₜ was measured in a Petri dish experiment at equal conditions. The reactor improves the growth rate of the fungus by 270%. The reactor leads to a higher mass transfer by continuously pumping the medium in the recycle and by actively supplying the culture with air.

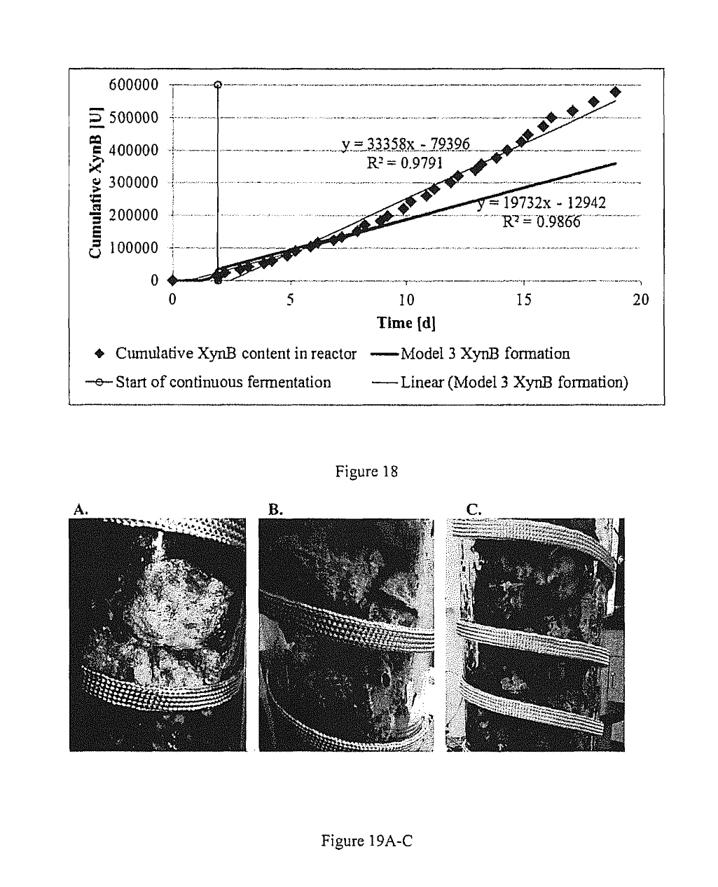

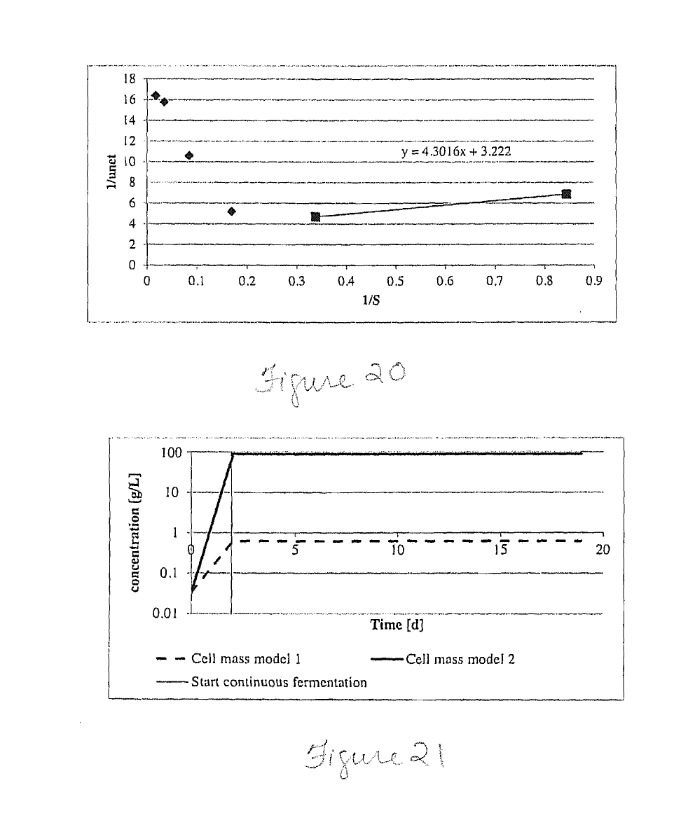

An alternative model (Model 3) could be established using the XynB productivity determined in previously. The productivity for a culture grown on medium without pyridoxine was 21.14 U/g dry cell mass*h. The predicted dry cell mass was calculated in the same manner as in Model 2. The corresponding XynB prediction was calculated using the productivity and multiplying it with the predicted cell mass and time. The resulting predicted XynB content is given as the accumulative XynB content in U. The comparison of the predicted and actual accumulative XynB content can be seen in FIG. 18. The predicted XynB formation over time follows a lower slope than the actual XynB production in the reactor. After completion of 18 days continuous fermentation, the total cell mass inside the reactor was measured. While Model 2 predicted a cell mass 61 g, the total cell mass after 18 days was 149 g. It is unknown what percentage of the total cell mass was viable cell mass. Furthermore, it was observed that the cell mass in the reactor increased over time. FIG. 19A-C shows the reactor column at different time points. At day 7 a small amount of pyridoxine solution was added in order to keep XynB production running. It is possible that some of the pyridoxine recycled through the reactor over a prolonged time, allowing the fungus to grow slowly but steadily. No more pyridoxine injection was necessary after day 7 for the rest of the test period. The pH was relatively constant over 11 days, signaling that the fungus was able to utilize pyridoxine. A more likely explanation can be found in a process called autolysis. It was shown that filamentous fungi under starvation initiate a process where in order to allow mycelium tip growth, old mycelia are broken down enzymatically to recycle cell components (White et al. 2002). This recycle of old mycelia mass could be a potential source for recycled pyridoxine and would explain why the fungal mass, viable plus dead, increases over time. A slow increase of cell mass over time would also explain the higher accumulative XynB content compared to the model prediction.

It is proposed that an A. nidulans mutant with a different marker, which is not essential for amino acid metabolism, would produce higher amounts of protein compared to the pyridoxine system. A potential marker could be biotin. Biotin is used for carboxylation reactions and its absence would not interfere with amino acid metabolism, but limit growth. Biotin is a coenzyme for pyruvate, acetyl-CoA, and propionyl-CoA carboxylase (Voet and Voet 2004). When acetyl-CoA carboxylase is inactive due to biotin depletion, no fatty acid synthesis would take place. It is assumed that growth would be limited, since membrane wall components (fatty acids) are missing.

### CONCLUSION

It was demonstrated that an embodiment of a trickle bed reactor system is applicable in producing enzymes by A. nidulans. Fungal mycelia favor surfaces for growth and can clog pipes, tubing tube connectors, reactor inlets and outlets. A solution to these problems is limiting the growth of the organism by changing the conditions. This was successfully demonstrated by limiting the pyridoxine supply to an A. nidulans mutant unable to synthesize its own pyridoxine over a period of 19 days. Small amounts of pyridoxine have to be added to the system to elongate enzyme productivity.

### Example 3

### Limiting Other Nutrients

The pyridoxine limitation system has shown to be a successful method of limiting growth while enzyme production continue. As discussed earlier, pyridoxine is essential for amino acid metabolism. As pyridoxine is a marker for this particular A. nidulans mutant, other coenzymes can also act as a marker. In an exemplary aspect, biotin is the coenzyme whose limitation improves the client protein formation.

Biotin is involved in carboxylation reactions. Pyruvate carboxylase, acetyl-CoA carboxylase, and propionyl-CoA use biotin as a CO₂ carrier (Voet and Voet 2004). Pyruvate carboxylase initiates the first step in gluconeogenesis by converting pyruvate to oxalacetate. Acetyl-CoA carboxylase is involved in fatty acid synthesis and propionyl-CoA is involved in odd-chain fatty acid oxidation (Voet and Voet 2004). Fatty acids are required for cell membrane synthesis. The limitation of biotin for an A. nidulans mutant with a biotin marker limits growth but does not affect enzyme production. Continuous client enzyme production and the limitation of growth over time are determined. Limiting biotin leads to even higher client protein productivity compared to system with a pyridoxine marker because amino acid metabolism is not affected by the limitation of biotin.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art.

While the inventive device has been described and illustrated herein by reference to certain embodiments in relation to the drawings attached hereto, various changes and further modifications, apart from those shown or suggested herein, may be made therein by those skilled in the art, without departing from the spirit of the inventive concept, the scope of which is to be determined by the following claims.

### REFERENCES

Adams T H, Timberlake W E. 1990. Developmental repression of growth and gene expression in Aspergillus. Proceedings of the National Academy of Sciences 87(14):5405.
Aguilar C N, Augur C, Favela-Torres E, Viniegra-González G. 2001. Production of tannase by Aspergillus niger Aa-20 in submerged and solid state fermentations: influence of glucose and tannic acid. Journal of Industrial Microbiology and Biotechnology 26(5):296-302.
Azin M, Moravej R, Zareh D. 2007. Production of xylanase by Trichoderma longibrachiatum on a mixture of wheat bran and wheat straw: Optimization of culture condition by Taguchi method. Enzyme and microbial technology 40(4):801-805.
Betini J H A, Michelin M, Peixoto-Nogueira S C, Jorge J A, Terenzi H F, Polizeli M. 2009. Xylanases from Aspergillus niger, Aspergillus niveus and Aspergillus ochraceus produced under solid-state fermentation and their application in cellulose pulp bleaching. Bioprocess and biosystems engineering 32(6):819-824.
Boender L G M, De Hulster E A F, Van Maris A J A, Daran-Lapujade P A S, Pronk J T. 2009. Quantitative physiology of Saccharomyces cerevisiae at near-zero specific growth rates. Applied and environmental microbiology 75(17):5607.
Bradford M M. 1976. A rapid and sensitive method for the quantitation of microgram quantities of protein utilizing the principle of protein-dye binding. Analytical Biochemistry 72(1-2):248-254.
Broderick A, Greenshields R. 1981. Sporulation of Aspergillus niger and Aspergillus ochraceus in continuous submerged liquid culture. Journal of general microbiology 126(1):193.
Cannel E, Moo-Young M. 1980. Solid state fermentation systems. Process Biochemistry 15:2-7.
Cheng K C, Demirci A, Catchmark J M. 2010. Advances in biofilm reactors for production of value-added products. Applied microbiology and biotechnology 87(2):445-456.
de Carvalho Peixoto-Nogueira S, Michelin M, Betini J H A, Jorge J A, Terenzi H F, de Lourdes Teixeira de Moraes Polizeli M. 2009. Production of xylanase by Aspergilli using alternative carbon sources: application of the crude extract on cellulose pulp biobleaching. Journal of Industrial Microbiology and Biotechnology 36(1):149-155.
Diaz-Godinez G, Soriano-Santos J, Augur C, Viniegra-Gonzalez G. 2001. Exopectinases produced by Aspergillus niger in solid-state and submerged fermentation: a comparative study. Journal of Industrial Microbiology and Biotechnology 26(5):271-275.
Durand A, Almanza S, Renaud R, Maratray J. 1997. Solid state fermentations: an attractive alternative to submerged-liquid fermentations. Agro Food Industry Hi-Tech 8:39-42.
El-Enshasy H, Hellmuth K, Rinas U. 1999. Fungal morphology in submerged cultures and its relation to glucose oxidase excretion by recombinant Aspergillus niger. Applied biochemistry and biotechnology 81(1):1-11.
Fujita M, Iwahori K, Tatsuta S, Yamakawa K. 1994. Analysis of pellet formation of Aspergillus niger based on shear stress. Journal of fermentation and bioengineering 78(5):368-373.
Galbraith J C, Smith J. 1969. Sporulation of Aspergillus niger in submerged liquid culture. Journal of general microbiology 59(1):31.
Garcia-Kirchner O, Mu\ddot{n}oz-Aguilar M, Pérez-Villalva R, Huitron-Vargas C. 2002. Mixed submerged fermentation with two filamentous fungi for cellulolytic and xylanolytic enzyme production. Applied biochemistry and biotechnology 98(1):1105-1114.
Gessesse A, Mamo G. 1999. High-level xylanase production by an alkaliphilic Bacillus sp. by using solid-state fermentation. Enzyme and microbial technology 25(1-2):68-72.
Jørgensen T R, Nitsche B M, Lamers G E, Arentshorst M, van den Hondel C A, Ram A F. 2010. Transcriptomic insights into the physiology of Aspergillus niger approaching a specific growth rate of zero. Applied and environmental microbiology 76(16):5344-5355.
Lonsane B K, Ghidyal N P G, Budiatman S, Ramakrishna S V. 1985. Engineering aspects of solid-state fermentation. Enzyme and Microbial Technology 7:258-265.
Mitchell D, Berovic M, Krieger N. 2000. Biochemical engineering aspects of solid state bioprocessing. New Products and New Areas of Bioprocess Engineering: 61-138.
Mitchell D A, Lonsane B K. 1992. Definition, characteristics and potential. In: Doelle H W, Mitchell D A, Rolz C E, editors. Solid substrate cultivation. London: Elsevier Science Publishers.
Ng A, Smith J, McIntosh A. 1972. Conidiation of Aspergillus niger in continuous culture. Archives of Microbiology 88(2):119-126.
Nigam P, Robinson T, Singh D. 2004. Solid-state fermentation: an overview. In: Arora D K, editor. Handbook of fungal biotechnology. New York: Marcel Dekker, Inc. Novozyme. 2010. Fermentation. Novozyme.
Park Y, Kang S, Lee J, Hong S, Kim S. 2002. Xylanase production in solid state fermentation by Aspergillus niger mutant using statistical experimental designs. Applied microbiology and biotechnology 58(6):761-766.
Pirt S. 1965. The maintenance energy of bacteria in growing cultures. Proceedings of the Royal

Society of London. Series B, Biological Sciences: 224-231.
Prosser I J, Tough A J. 1991. Growth mechanisms and growth kinetics of filamentous microorganisms. Biotechnology 10(4):253-274.
Romero-Gomez S, Augur C, Viniegra-González G. 2000. Invertase production by Aspergillus niger in submerged and solid-state fermentation. Biotechnology Letters 22(15):1255-1258.
Schrickx J M, Krave A S, Verdoes J C, van den Hondel C A, Stouthamer A H, van Verseveld H W. 1993. Growth and product formation in chemostat and recycling cultures by Aspergillus niger N402 and a glucoamylase overproducing transformant, provided with multiple copies of the glaA gene. Journal of general microbiology 139(11):2801.
Schrickx J M, Raedts M J, Stouthamer A H, Verseveld H Wv. 1995. Organic acid production by Aspergillus niger in recycling culture analyzed by capillary electrophoresis. Analytical Biochemistry 231:175-181.
Segato F, Damásio A R L, Gonçalves T A, Lucas R C, Squina F M, Decker S, Prade R A. 2011. Secretion of client proteins in Aspergillus. Submitted.
Skromne I, Sánchez O, Aguirre J. 1995. Starvation stress modulates the expression of the Aspergillus nidulans brlA regulatory gene. Microbiology 141(1):21.
Sluiter A, Hames B, Ruiz R, Scarlata C, Sluiter J, Templeton D. 2008. Determination of sugars, byproducts, and degradation products in liquid fraction process samples. National Renewable Energy Laboratory (NREL/TP-510-42623).
Tunga R, Banerjee R, Bhattacharyya B C. 1999. Studies of some physical parameters for large scale protease production by SSF. Bioprocess Engineering 21:107-112.
Verseveld H W, Metwally M, Sayed M, Osman M, Schrickx J M, Stouthamer A H. 1991. Determination of the maximum product yield from glucoamylase-producing Aspergillus niger grown in the recycling fermentor. Antonie van Leeuwenhoek 60(3):313-323.
Viniegra-González G, Favela-Torres E, Aguilar C N, Rómero-Gomez S J, Díaz-Godínez G, Augur C. 2003. Advantages of fungal enzyme production in solid state over liquid fermentation systems. Biochemical Engineering Journal 13(2-3):157-167.
Viniegra-González G, Favela-Torres E. 2004. Basic principles for the production of fungal enzymes by solid-state fermentation. In: Arora D K, editor. Handbook of fungal biotechnology. New York: Marcel Dekker, Inc.
Voet D, Voet J G. 2004. Biochemistry. Hoboken: John Wiley & Sons, Inc. Vogel H F. 1964. Distribution of lysine pathways among fungi: evolutionary implications. American Naturalist 98:435-446.
White S, McIntyre M, Berry D R, McNeil B. 2002. The autolysis of industrial filamentous fungi. Critical reviews in biotechnology 22(1):1-14.
Winterbottom J M, King M. 1999. Reactor design for chemical engineers: CRC.
\check{Z}nidar\check{s}i\check{c} P, Komel R, Pavko A. 2000. Influence of some environmental factors on Rhizopus nigricans submerged growth in the form of pellets. World Journal of Microbiology and Biotechnology 16(7):589-593.

What is claimed is:

1. A method of producing a product of interest using a filamentous fungus, comprising
providing a filamentous fungus that is auxotrophic for at least one nutrient that is necessary for growth of the filamentous fungus but which is not necessary for synthesis of the product of interest, and
culturing the filamentous fungus in the absence of the at least one nutrient.

2. The method of claim 1, wherein said filamentous fungus is Aspergillus nidulans that is auxotrophic for pyroxidine.

3. The method of claim 1, wherein said product of interest is a protein.

4. The method of claim 3, wherein said protein is an enzyme.

5. The method of claim 4, wherein said enzyme is xylanase B (XynB).

6. The method of claim 1, wherein said method is carried out in a trickle bed reactor (TBR).
